SEQUENCE LISTING (40 sequences)

SEQ ID NO: 1 - DNA, 18 bp, Artificial Sequence
  (Synthetic)Human b-actin_F
gtcttcccctccatcgtg

SEQ ID NO: 2 - DNA, 20 bp, Artificial Sequence
  (Synthetic)Human b-actin_R
aggtgtggtgccagattttc

SEQ ID NO: 3 - DNA, 20 bp, Artificial Sequence
  (Synthetic)Human ALDH1A1_F
cgccagacttacctgtccta

SEQ ID NO: 4 - DNA, 22 bp, Artificial Sequence
  (Synthetic)Human ALDH1A1_R
gtcaacatcctccttatctcct

SEQ ID NO: 5 - DNA, 20 bp, Artificial Sequence
  (Synthetic)Human ALDH1A2_F
cagctttgtgctgtggcaat

SEQ ID NO: 6 - DNA, 20 bp, Artificial Sequence
  (Synthetic)Human ALDH1A2_R
ggaaagccagcctccttgat

SEQ ID NO: 7 - DNA, 24 bp, Artificial Sequence
  (Synthetic)Human EpCAM_F
agttggtgcacaaaatactgtcat

SEQ ID NO: 8 - DNA, 20 bp, Artificial Sequence
  (Synthetic)Human EpCAM_R
tcccaagttttgagccattc

SEQ ID NO: 9 - DNA, 20 bp, Artificial Sequence
  (Synthetic)Human CD44_F
tccaacacctcccagtatga

SEQ ID NO: 10 - DNA, 20 bp, Artificial Sequence
  (Synthetic)Human CD44_R
ggcaggtctgtgactgatgt

SEQ ID NO: 11 - DNA, 18 bp, Artificial Sequence
  (Synthetic)Human CD90_F
agagacttggatgaggag

SEQ ID NO: 12 - DNA, 18 bp, Artificial Sequence
  (Synthetic)Human CD90_R
ctgagaatgctggagatg

SEQ ID NO: 13 - DNA, 22 bp, Artificial Sequence
  (Synthetic)Human CD133_F
accaggtaagaacccggatcaa

SEQ ID NO: 14 - DNA, 23 bp, Artificial Sequence
  (Synthetic)Human CD133_R
caagaattccgcctcctagcact

SEQ ID NO: 15 - DNA, 23 bp, Artificial Sequence
  (Synthetic)Human LGR5_F
cctgcttgactttgaggaagacc

SEQ ID NO: 16 - DNA, 22 bp, Artificial Sequence
  (Synthetic)Human LGR5_R
ccagccatcaagcaggtgttca

SEQ ID NO: 17 - DNA, 25 bp, Artificial Sequence
  (Synthetic)Human Oct3/4_F
cttgctgcagaagtgggtggaggaa

SEQ ID NO: 18 - DNA, 21 bp, Artificial Sequence
  (Synthetic)Human Oct3/4_R
ctgcagtgtgggtttcgggca

SEQ ID NO: 19 - DNA, 21 bp, Artificial Sequence
  (Synthetic)Human Sox2_F
catcacccacagcaaatgaca

SEQ ID NO: 20 - DNA, 24 bp, Artificial Sequence
  (Synthetic)Human Sox2_R
gctcctaccgtaccactagaactt

SEQ ID NO: 21 - DNA, 23 bp, Artificial Sequence
  (Synthetic)Human Nanog_F
aatacctcagcctccagcagatg

SEQ ID NO: 22 - DNA, 24 bp, Artificial Sequence
  (Synthetic)Human Nanog_R
tgcgtcacaccattgctattcttc

SEQ ID NO: 23 - DNA, 25 bp, Artificial Sequence
  (Synthetic)Human ABCB1_F
tgacatttattcaaagttaaaagca

SEQ ID NO: 24 - DNA, 25 bp, Artificial Sequence
  (Synthetic)Human ABCB1_R
tagacactttatgcaaacatttcaa

SEQ ID NO: 25 - DNA, 20 bp, Artificial Sequence
  (Synthetic)Human ABCB2_F
cgttgtcagttatgcagcgg

SEQ ID NO: 26 - DNA, 20 bp, Artificial Sequence
  (Synthetic)Human ABCB2_R
atagatcccgtcacccacga

SEQ ID NO: 27 - DNA, 20 bp, Artificial Sequence
  (Synthetic)Human ABCB5_F
cacaaaaggccattcaggct

SEQ ID NO: 28 - DNA, 21 bp, Artificial Sequence
  (Synthetic)Human ABCB5_R
gctgaggaatccacccaatct

SEQ ID NO: 29 - DNA, 22 bp, Artificial Sequence
  (Synthetic)Human ABCC1_F
ggaataccagcaaccccgactt

SEQ ID NO: 30 - DNA, 23 bp, Artificial Sequence
  (Synthetic)Human ABCC1_R
ttttggttttgttgagaggtgtc

SEQ ID NO: 31 - DNA, 25 bp, Artificial Sequence
  (Synthetic)Human ABCG2_F
tcatgttaggattgaagccaaaggc

SEQ ID NO: 32 - DNA, 24 bp, Artificial Sequence
  (Synthetic)Human ABCG2_R
tgtgagattgaccaacagacctga

SEQ ID NO: 33 - DNA, 20 bp, Artificial Sequence
  (Synthetic)Human DKK1_F
tcccctgtgattgcagtaaa

SEQ ID NO: 34 - DNA, 20 bp, Artificial Sequence
  (Synthetic)Human DKK1_R
tccaagagatccttgcgttc

SEQ ID NO: 35 - DNA, 20 bp, Artificial Sequence
  (Synthetic)Human b-catenin_F
acagctcgttgtaccgctgg

SEQ ID NO: 36 - DNA, 20 bp, Artificial Sequence
  (Synthetic)Human b-catenin_R
agcttggggtccaccactag

SEQ ID NO: 37 - DNA, 20 bp, Artificial Sequence
  (Synthetic)Human AXIN2_F
agtgtgaggtccacggaaac

SEQ ID NO: 38 - DNA, 20 bp, Artificial Sequence
  (Synthetic)Human AXIN2_R
cttcacactgcgatgcattt

SEQ ID NO: 39 - DNA, 21 bp, Artificial Sequence
  (Synthetic)Human MMP-2_F
tctcctgacattgaccttggc

SEQ ID NO: 40 - DNA, 22 bp, Artificial Sequence
  (Synthetic)Human MMP-2_R
caaggtgctggctgagtagatc

(19) United States
(12) Patent Application Publication
(10) Pub. No.: US 2021/0371827 A1
(43) Pub. Date: Dec. 2, 2021

(54) METHOD FOR PREPARING CANCER STEM CELL SPHEROIDS

(71) Applicant: KOREA ADVANCED INSTITUTE OF SCIENCE AND TECHNOLOGY, Yuseong-gu, Daejeon (KR)

(72) Inventors: Sang Yong JON, Yuseong-gu, Daejeon (KR); Min Suk CHOI, Yuseong-gu, Daejeon (KR); Sung Gap IM, Yuseong-gu, Daejeon (KR); Daeyoup LEE, Yuseong-gu, Daejeon (KR); Seung Jung YU, Yuseong-gu, Daejeon (KR); Yoon Jung CHOI, Yuseong-gu, Daejeon (KR)

(21) Appl. No.: 16/963,278

(22) PCT Filed: Nov. 13, 2018
(86) PCT No.: PCT/KR2018/013838

§ 371 Date: Jul. 20, 2020

(30) Foreign Application Priority Data
Jan. 31, 2018 (KR) 10-2018-0012338
Oct. 25, 2018 (KR) 10-2018-0128190

(51) Int. Cl. C12N 5/095; C12N 5/00; A61K 35/13

(52) U.S. Cl. CPC C12N 5/0695; C12N 5/0018; C12N 2501/998; C12N 2500/50; A61K 35/13

(57) ABSTRACT

The present invention relates to a method or kit for producing cancer stem cell spheroids, and a method of screening of drugs for treating cancer cell resistance using the prepared cancer stem cell spheroid, and it can conveniently produce cancer stem cell spheroids, and the prepared cancer stem cell spheroids can be effectively utilized for screening drugs for treating cancer cell resistance.

### TECHNICAL FIELD

The present invention relates to a method or a kit for producing cancer stem cell spheroids. In addition, it relates to a method of screening of drugs for treating cancer cell resistance using cancer stem cell spheroids prepared by the method or kit.

### BACKGROUND ART

Cancer stem cells (CSCs or tumor-initiating cells: TIC) have many features similar to normal stem cells, such as self-regenerative ability, endogenous drug resistance and differentiation, and the like. Since cancer cells similar to stem cells have been discovered in acute myeloid leukemia, there is increasing evidence that a small number of cancer stem cells are present in tumor aggregates primarily responsible for tumor recurrence and drug resistance. Therefore, cancer stem cells have attracted considerable attention in the field of cancer research and drug resistance.

The cancer stem cells are generally isolated from patient-derived tumor tissue based on cancer stem cell surface markers. However, the supply of the patient-derived tumor tissue is limited, and only a small amount of cancer stem cells can be isolated, which makes it difficult to obtain cancer stem cells. Alternatively, attempts have been made to separate cancer stem cells from existing cancer cell lines, but since cancer stem cells are contained less than 1 to 2% in the cancer cell line, it is not practical to secure a sufficient amount of cancer stem cells (Cell 144, 646-674 (2011)). In addition, since the three-dimensional structure of cancer cells can better represent the tumor environment than the two-dimensional monolayer structure, considerable interest is currently shown in developing a method for promoting formation of cancer cells. The spheroid, which is used for drug screening or efficacy testing, is currently produced by a method for inserting cells into a hole of a hydrophilic ULA (ultra-low-attachment) surface, a concave agarose gel (U-bottom) or a hanging-drop cell substrate, and the like. However, even the spheroid produced by the method does not sufficiently contain cancer stem cells. In this situation, there is a need to develop a simple method for producing cancer stem cell spheroids having cancer-formation ability in a human cancer cell line.

Accordingly, the present inventors have tried to develop a method for producing cancer stem cell spheroids, and as a result, they have established a method for producing cancer stem cell spheroids using a cell culture substrate comprising a cyclosiloxane polymer and a medium comprising albumin, thereby completing the present invention.

### Technical Problem

One object of the present invention is to provide a composition for inducing cancer stem cells from cancer cells, comprising albumin and a medium for cell culture.

Another object of the present invention is to provide a method of producing cancer stem cells from cancer cells, comprising a step of culturing cancer cells using a composition for inducing cancer stem cells from cancer cells, comprising a medium comprising albumin.

Other object of the present invention is to provide a kit for producing cancer stem cell spheroids, comprising a composition for inducing cancer stem cells from cancer cells, comprising albumin and a medium for cell culture, wherein the cell culture substrate comprises a cyclosiloxane polymer, and the medium comprises albumin.

Other object of the present invention is to provide a screening of drugs for treating cancer cell resistance, comprising (a) preparing cancer stem cell spheroids by the method of producing cancer stem cell spheroids; (b) treating a candidate substance for treating cancer cell resistance to the cancer stem cell spheroid of the (a) step; and (c) comparing cancer stem cell spheroids group in which the candidate substance for treating cancer cell resistance of the (b) step and a control group in which the candidate substance for treating cancer cell resistance is not treated.

### Technical Solution

This is specifically described as follows. Meanwhile, each description and embodiment disclosed in the present application may be applied to each other description and embodiment. In other words, all combinations of various elements disclosed in the present application fall within the scope of the present application. In addition, the scope of the present application is not considered to be limited by the specific description disclosed below.

As one aspect to achieve the objects of the present invention, a composition for inducing cancer stem cells from cancer cells, comprising a medium for cell culture containing albumin is provided.

As another aspect to achieve the objects of the present invention, a method for preparing cancer stem cells from cancer cells, comprising culturing cancer cells using a composition for inducing cancer stem cells from cancer cells, comprising a medium for cell culture containing albumin is provided.

As other aspect to achieve the objects of the present invention, a kit for preparing cancer stem cell spheroids, comprising a cell culture substrate, and a composition for inducing cancer stem cells from cancer cells, comprising a medium for cell culture containing albumin, wherein the cell culture substrate comprise a cyclosiloxane polymer and the medium comprises albumin, is provided.

As other aspect to achieve the objects of the present invention, a method of screening of drugs for treating cancer cell resistance, comprising preparing cancer stem cell spheroids; treating a candidate substance for treating cancer cell resistance to the cancer stem cell spheroid; and comparing cancer stem cell spheroids group in which the candidate substance for treating cancer cell resistance is treated and a control group in which the candidate substance for treating cancer cell resistance is not treated is provided.

The present inventors have found that when cancer cells are cultured in a medium comprising albumin, on a cell culture substrate comprising a polymer formed by a cyclosiloxane compound, a three-dimensional cancer stem cell spheroid like in vivo environment, which completely has characteristics of the cancer stem cell, can be prepared with high yield, thereby providing the invention.

Hereinafter, the present invention will be described in more detail.

As one aspect to achieve the objects of the present invention, a method for producing cancer stem cells from cancer cells, comprising culturing cancer cells using a composition for inducing cancer stem cells from cancer cells, comprising a medium for cell culture containing albumin is provided.

The culturing cancer cells using a composition for inducing cancer stem cells from cancer cells, comprising a medium for cell culture containing albumin is culturing an isolated cancer cell using a composition comprising a medium for cell culture comprising albumin, and the culturing may be performed on a cell culture substrate comprising a cyclosiloxane polymer.

The term of the present invention, “cancer cell” or “isolated cancer cell” may be a cell derived from a human or a cell derived from various individuals except for humans, but not limited thereto. In addition, the isolated cancer cell may include all of in vivo or in vitro cells, but not limited thereto. Specifically, the isolated cancer cell may be specifically a cell derived from various tissues of humans, and may be cancer cells derived from ovarian cancer, breast cancer, liver cancer, brain cancer, colorectal cancer, prostate cancer, cervical cancer, lung cancer, stomach cancer, skin cancer, pancreatic cancer, oral cancer, rectal cancer, laryngeal cancer, thyroid cancer, parathyroid cancer, colon cancer, bladder cancer, peritoneal carcinoma, adrenal cancer, tongue cancer, small intestine cancer, esophageal cancer, renal pelvis cancer, renal cancer, heart cancer, duodenal cancer, ureteral cancer, urethral cancer, pharynx cancer, vaginal cancer, tonsil cancer, anal cancer, pleura cancer, thymic carcinoma or nasopharyngeal carcinoma, but not limited thereto, and it includes all cancer cells which can be used for the objects of the present invention, and includes all primary cultured cells isolated by biopsy from cancer tissue, or established cell lines, but not limited thereto.

In addition, to confirm the cancer cell, a cancer cell marker may be used. Specifically, as the marker, AFP (Alpha-fetoprotein), CA15-3, CA27-29, CA19-9, CA-125, Calcitonin, Calretinin, CD34, CD117, Desmin, inhibin, Myo D1, NSE (neuronspecific enolase), PLAP (placental alkaline phosphatase) and PSA (prostatespecific antigen) may be used, but not limited thereto.

The term of the present invention, “cyclosiloxane compound” is used to include compounds which have a cyclosiloxane structure as a basic structure, and has a functional group (for example, alkyl group, alkenyl group, etc.) at the position of its silicon atom. According to one embodiment of the present invention, the cyclosiloxane compound is represented by the following chemical formula 1.

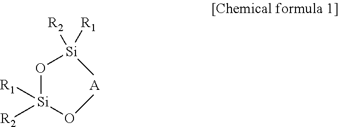

In the formula, A is

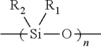

(n=an integer of 1-8); and

R1 is independently of each other hydrogen or C2-10 alkenyl with the proviso that at least two positions of R1 are C2-10 alkenyl; and

R2 is independently of each other hydrogen, C1-10 alkyl, C2-10 alkenyl, halo group, metal element, C5-14 heterocycle, C3-10 cycloalkyl or C3-10 cycloalkenyl.

The term of the present invention, “alkyl” means a straight-chain or branched-chain, unsubstituted or substituted, saturated hydrocarbon group, and for example, includes methyl, ethyl, propyl, isobutyl, pentyl or hexyl, and the like. C1-C10 alkyl means an alkyl group having an alkyl unit of 1 to 10 carbon atoms, and when C1-C10 alkyl is substituted, the number of carbon atoms of the substituent is not comprised.

According to one embodiment of the present invention, herein, C1-C10 alkyl is C1-C8 alkyl, C1-C7 alkyl or C1-C6 alkyl. The term of the present invention, “alkenyl” represents a straight-chain or branched-chain, unsubstituted or substituted, unsaturated hydrocarbon group having designated carbon atoms, and for example, includes vinyl, propenyl, allyl, isopropenyl, butenyl, isobutenyl, t-butenyl, n-pentenyl, and n-hexenyl. C2-C10 alkenyl means an alkenyl group having an alkenyl unit of 1 to 10 carbon atoms, and when C2-C10 alkenyl is substituted, the number of carbon atoms of the substituent is not comprised.

According to one embodiment of the present invention, herein, C2-10 alkenyl is C2-8 alkenyl, C2-6 alkenyl, C2-5 alkenyl, C2-4 alkenyl or C2-3 alkenyl. According to one embodiment of the present invention, at least three parts of the R1 is C2-10 alkenyl. According to one embodiment of the present invention, the cyclosiloxane has n+1 or n+2 of C2-10 alkenyl at the R1 position. For example, when n is 2, the compound of chemical formula 1 becomes a cyclotetrasioloxane having 3 or 4 C2-10 alkenyls at the R1 position. This alkenyl group is involved in polymerization.

The term of the present invention, “halo” represents a halogen element, and for example, includes flouro, chloro, bromo and iodo. The term of the present invention, “metal element” means an element which makes metallic simple substance such as alkali metal elements (Li, Na, K, Rb, Cs, Fr), alkali earth metal elements (Ca, Sr, Ba, Ra), aluminum family elements (Al, Ga, In, Tl), tin family elements (Sn, Pb), coinage metal elements (Cu, Ag, Au), zinc family elements (Zn, Cd, Hg), rare earth elements (Sc, Y, 57-71), titanium family elements (Ti, Zr, Hf), vanadium family elements (V, Nb, Ta), chrome family elements (Cr, Mo, W), manganese family elements (Mn, Tc, Re), iron family elements (Fe, Co, Ni), platinum family elements (Ru, Rh, Pd, Os, Ir, Pt) and actinide elements (89-103), and the like.

The term of the present invention, “heterocycle” means a partially or completely saturated, monocycle type or bicycle type of 5 to 14 membered heterocycle ring. N, O and S are examples of heteroatoms. Pyrrole, furan, thiophene, imidazole, pyrazole, oxazole, isoxazole, thiazole, isothiazole, tetrazole, 1,2,3,5-oxathiadiazole-2-oxide, triazolone, oxadiaxolone, isoxazolone, oxadiazolidine dione, 3-hydroxypyro-2,4-dione, 5-oxo-1,2,4-thiadiazole, pyridine, pyrazine, pyrimidine, indole, isoindole, indazole, phthalazine, quinoline, isoquinoline, quinoxaline, quinazoline, cinnoline and carboline are examples of C5-14 heterocycles.

The term of the present invention, “cycloalkyl” means a cyclic hydrocarbon radical, and this includes cyclopropyl, cyclobutyl and cyclopentyl. C3-10 cycloalkyl means a cycloalkyl having 3-10 carbon atoms which form a ring structure, and when C3-10 cycloalkyl is substituted, the number of carbon atoms of the substituent is not comprised.

According to one embodiment of the present invention, herein, C1-C10 cycloalkyl is C1-C8 cycloalkyl, C1-C7 cycloalkyl or C1-C6 cycloalkyl.

The term of the present invention, “cycloalkenyl” means a cyclic hydrocarbon group having at least one double bond, and for example, includes cyclopentene, cyclohexene and cyclohexadiene. C3-10 cycloalkenyl means a cycloalkenyl having 3-10 carbon atoms which form a ring structure, and when C3-10 cycloalkenyl is substituted, the number of carbon atoms of the substituent is not comprised.

According to one embodiment of the present invention, C2-10 cycloalkenyl is C2-8 cycloalkenyl, C2-6 cycloalkenyl, C2-5 cycloalkenyl, C2-4 cycloalkenyl or C2-3 cycloalkenyl.

According to one embodiment of the present invention, the R2 is independently of each other hydrogen, C1-10 alkyl or C2-10 alkenyl. According to one specific example, at least two parts or at least three parts of the R2 may be C1-10 alkyl or C2-10 alkenyl. According to one specific example, the cyclosiloxane may have n+1 or n+2 of C1-10 alkyl or C2-10 alkenyl at the R2 position.

According to one embodiment of the present invention, the n is an integer of 1-7, an integer of 1-6, an integer of 1-5, an integer of 1-4 or an integer of 1-3.

According to one embodiment of the present invention, the cyclosiloxane compound is selected from the group consisting of 2,4,6,8-tetra(C2-10)alkenyl-2,4,6,8-tetra(C1-10)alkylcyclotetrasiloxane, 1,3,5-tri(C1-10)alkyl-1,3,5-tri(C2-10)alkenylcyclotrisiloxane, 1,3,5,7-tetra(C1-10)alkyl-1,3,5,7-tetra(C2-10)alkenylcyclotetrasiloxane, 1,3,5,7,9-penta(C1-10)alkyl-1,3,5,7,9-penta(C2-10)alkenylcyclopentasiloxane, 1,3,5-tri(C1-10)alkyl-1,3,5-tri(C2-10)alkenylcyclotrisiloxane, 1,3,5,7-tetra(C1-10)alkyl-1,3,5,7-tetra(C2-10)alkenylcyclotetrasiloxane, 1,3,5,7,9-penta(C1-10)alkyl-1,3,5,7,9-penta(C2-10)alkenylcyclopentasiloxane, 1,3,5-tri(C1-10)alkyl-1,3,5-tri(C2-10)alkenylcyclotrisiloxane, 1,3,5,7-tetra(C1-10)alkyl-1,3,5,7-tetra(C2-10)alkenylcyclotetrasiloxane, 1,3,5,7,9-penta(C1-10)alkyl-1,3,5,7,9-penta(C2-10)alkenylcyclopentasiloxane, hexa(C2-10)alkenylcyclotrisiloxane, octa(C2-10)alkenylcyclotetrasiloxane, deca(C2-10)alkenylcyclopentasiloxane, 2,4,6,8-tetravinyl-2,4,6,8,-tetramethylcyclotetrasiloxane and combinations thereof.

According to one specific example, the cyclosiloxane compound is selected from the group consisting of 1,3,5-trivinyl-1,3,5-trimethylecyclotrisiloxane, 2,4,6,8-tetramethyl-2,4,6,8-tetravinylcyclotetrasiloxane (V4D4), 2,4,6,8,10-pentamethyl-2,4,6,8,10-pentavinylcyclopentasiloxane, 2,4,6,8,10,12-hexamethyl-2,4,6,8,10,12-hexavinyl-cyclohexasiloxane, octa(vinylsilasesquioxane), 2,2,4,4,6,6,8,8,10,10,12,12-dodecamethylcyclohexasiloxane, 2,4,6,8-tetra(C2-4)alkenyl-2,4,6,8-tetra(C1-6)alkylcyclotetrasiloxane (as one example, 2,4,6,8-tetravinyl-2,4,6,8-tetramethylcyclotetrasiloxane), 1,3,5-tri(C1-6)alkyl-1,3,5-tri(C2-4)alkenylcyclotrisiloxane (as one example, 1,3,5-triisopropyl-1,3,5-trivinylcyclotrisiloxane), 1,3,5,7-tetra(C1-6)alkyl-1,3,5,7-tetra(C2-4)alkenylcyclotetrasiloxane (as one example, 1,3,5,7-tetraisopropyl-1,3,5,7-tetravinylcyclotetrasiloxane), 1,3,5,7,9-penta(C1-6)alkyl-1,3,5,7,9-penta(C2-4)alkenylcyclopentasiloxane (as one example, 1,3,5,7,9-pentaisopropyl-1,3,5,7,9-pentavinylcyclopentasiloxane), 1,3,5-tri(C1-6)alkyl-1,3,5-tri(C2-4)alkenylcyclotrisiloxane (as one example, 1,3,5-tri-sec-butyl-1,3,5-trivinylcyclotrisiloxane), 1,3,5,7-tetra(C1-6)alkyl-1,3,5,7-tetra(C2-4)alkenylcyclotetrasiloxane (as one example, 1,3,5,7-tetra-sec-butyl-1,3,5,7-tetravinylcyclotetrasiloxane), 1,3,5,7,9-penta(C1-6)alkyl-1,3,5,7,9-penta(C2-4)alkenylcyclopentasiloxane (as one example, 1,3,5,7,9-penta-sec-butyl-1,3,5,7,9-pentavinylcyclopentasiloxane), 1,3,5-tri(C1-6)alkyl-1,3,5-tri(C2-4)alkenylcyclotrisiloxane (as one example, 1,3,5-triethyl-1,3,5-trivinylcyclotrisiloxane), 1,3,5,7-tetra(C1-6)alkyl-1,3,5,7-tetra(C2-4)alkenylcyclotetrasiloxane (as one example, 1,3,5,7-tetraethyl-1,3,5,7-tetravinylcyclotetrasiloxane), 1,3,5,7,9-penta(C1-6)alkyl-1,3,5,7,9-penta(C2-4)alkenylcyclopentasoxane (as one example, 1,3,5,7,9-pentaethyl-1,3,5,7,9-pentavinylcyclopentasiloxane), hexa(C2-4)alkenylcyclotrisiloxane (as one example, hexavinylcyclotrisiloxane), octa(C2-4)alkenylcyclotetrasiloxane (as one example, octavinylcyclotetrasiloxane), deca(C2-4)alkenylcyclopentasiloxane (as one example, decavinylcyclopentasiloxane) and combinations thereof.

The term of the present invention, “cell culture substrate comprising a cyclosiloxane compound” may mean that a polymer formed by cyclosiloxane is a part of a cell culture substrate (for example, cell culture substrate of which surface is coated with the polymer), and also may mean that the solid polymer formed by cyclosiloxane itself may be used as a cell culture substrate, but not limited thereto.

It is sufficient for the cell culture substrate to provide any space capable of culturing a cell, and its shape is not limited. For example, the cell culture substrate may be a dish (culture dish), a chalet or plate (for example, 6-well, 24-well, 48-well, 96-well, 384-well or 9600-well microtiter plate, microplate, dip-well plate, etc.), a flask, a chamber slide, a tube, a cell factory, a roller bottle, a spinner flask, hollow fibers, a microcarrier, beads, and the like, but not limited thereto, and any material having support properties can be used without limitation as the cell culture substrate, and for example, plastics (for example, polystyrene, polyethylene, polypropylene, etc.), metals, silicon and glass, and the like may be used as the cell culture substrate.

In addition, the polymer formed by the cyclosiloxane compound is used as a meaning including all of (1) homopolymers formed by polymerization of homogeneous cyclosiloxane compounds, (2) copolymers formed by polymerization of heterogeneous cyclosiloxane compounds, and (3) copolymers formed by polymerization of homogeneous or heterogeneous cyclosiloxane compounds with other monomer compounds. Herein, the copolymer may be random copolymers, block copolymers, alternating copolymers or graft copolymers, but not limited thereto.

Therefore, according to one embodiment of the present invention, the polymer formed by the cyclosiloxane compound is a homogeneous polymer formed by polymerization of homogeneous cyclosiloxane compounds.

According to another embodiment of the present invention, the polymer formed by the cyclosiloxane compound is a copolymer formed by a first monomer that is the cyclosiloxane compound and a second monomer that can polymerize therewith.

According to one specific example, the second monomer is a cyclosiloxane compound different from the first monomer (copolymer formed by heterogeneous cyclosiloxane compounds).

According to another specific example, the second monomer is a compound having a carbon double bond for polymerization with the first monomer. Then, the first monomer may also have a carbon double bond for polymerization with the second monomer. Such a second monomer compound may be, for example, selected from the group consisting of siloxane having a vinyl group (for example, hexavinyldisiloxane, tetramethyldisiloxane, etc.), methacrylate-based monomers, acrylate-based monomers, aromatic vinyl-based monomers (for example, divinylbenzene, vinylbenzoate, styrene, etc.), acrylamide-based monomers (for example, N-isopropylacrylamide, N,N-dimethylacrylamide, etc.), maleic anhydride, silazane or cyclosilazane having a vinyl group (for example, 2,4,6-trimethyl-2,4,6-trivinylcyclosilazane, etc.), C3-10 cycloalkane having a vinyl group (for example, 1,2,4-trivinylcyclohexane, etc.), vinylpyrrolidone, 2-(methacryloyloxy)ethyl acetoacetate, 1-3(-aminopropyl)imidazole, vinylimidazole, vinylpyridine, silane having a vinyl group (for example, allyltrichlorosilane, acryloxymethyltrimethoxysilane, etc.) and combinations thereof.

According to other specific example, the second monomer may be at least one selected from the group consisting of 1,3,5-trivinyl-1,3,5-trimethylcyclotrisiloxane, 2,4,6,8-tetramethyl-2,4,6,8-tetravinylcyclotetrasiloxane (V4D4), 2,4,6,8,10-pentamethyl-2,4,6,8,10-pentavinylcyclopentasiloxane, 2,4,6,8,10,12-hexamethyl-2,4,6,8,10,12-hexavinyl-cyclohexasiloxane, octa(vinylsilasesquioxane), and 2,2,4,4,6,6,8,8,10,10,12,12-dodecamethyl cyclohexasiloxane.

The methacrylate-based monomer includes, for example, methacrylate, methacrylic acid, glycidyl methacrylate, perfluoromethacrylate, benzylmethacrylate, 2-(dimethylamino)ethylmethacrylate, perfurilmethacrylate, 3,3,4,4,5,5,6,6,7,7,8,8,9,9,10,10,10-heptadecaflourodecylmethacrylate, hexylmethacrylate, methacrylic anhydride, pentaflourophenylmethacrylate, propargylmethacrylate, tetrahydroperperillmethacrylate, butylmethacrylate, methacryloylchloride and di(ethyleneglycol)methylestermethacrylate, and the like.

The acrylate-based monomer includes, for example, acrylate, 2-(dimehtylamino)ethylacrylate, ethyleneglycolacrylate, 1H,1H,7H-dodecafluoroheptylacrylate, 1H,1H,7H-dodecafluoroheptylacrylate, isobornylacrylate, 1H,1H,2H,2H-perfluorodecylacrylate, tetrahydroperfurilacrylate, poly(ethyleneglycol)diacrylate, 1H,1H,7H-dodecafluoroheptylacrylate and propargylacrylate, and the like.

The copolymer of the present invention may further comprise a monomer other than monomers mentioned herein as a comonomer.

According to one embodiment of the present invention, the copolymer contains at least 50% or more of the cyclosiloxane compound. According to one specific example, the copolymer contains at least 60% or more, 70% or more, 80% or more or 90% or more of the cyclosiloxane compound. This content is based on the flow rate (unit:sccm), and 90% means the content of the cyclosiloxane compound contained in the copolymer formed by flowing (dropping) each monomer at a flow rate of 9:1 (cyclosiloxane compound:other monomer), and 80%, 70% and 60% mean the content of the cyclosiloxane compound comprised in the copolymer formed by flowing at a flow rate of 8:1, 7:1 and 6:1.

In addition, the cell culture substrate comprising the polymer may be a cell culture substrate comprising a polymer having various thicknesses. The thickness of the polymer may be, for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300 nm or more, or about 10,000, 5,000, 1,000, 900, 800, 700, 600, 500, 400, 300 nm or less, or about 10 to 300 nm, 10 to 500 nm, 10 to 1000 nm, 50 to 300 nm, 50 to 500 nm, 50-1000 nm, but not limited thereto.

In the method for preparing cancer stem cells from cancer cells, comprising culturing cancer cells using a composition for inducing cancer stem cells from cancer cells comprising a medium for cell culture containing albumin, the cancer stem cell may be in a spheroid form. The method may be characterized by not comprising any other compound known for additional gene manipulation or stem cell proliferation, or known to differentiate a stem cell from an adult cell. The medium for cell culture may not comprise other growth factors except for albumin.

The term, “spheroid” means a cell aggregate forming a three-dimensional sphere form by gathering 1000 or more of single cells, and as it can more accurately copy structural and physical properties of the three-dimensional tissue surrounding cells in a human body, it is usefully used in treatment and research fields, and on purpose of the present invention, the spheroid is characterized by cancer stem cells spheroid.

In addition, the term of the present invention, “cancer stem cell (or tumor initiating cell)” means a cell having an ability to produce tumor, and the cancer stem cell has similar characteristics to normal stem cells. The cancer stem cell causes tumor through self-regeneration and differentiation that are characteristics of the stem cell in various cell types, and therefore it has cancer-formation ability. It becomes a reason for recurrence and metastasis by causing new tumor distinguished from other groups in tumor by the cancer-formation ability. In addition, as another characteristic of the cancer stem cell, it has drug resistance, and therefore it has resistance to chemical therapies such as anticancer agent usage and the like, and thus only common cancer cells are removed and cancer stem cells remain without dying, and the cancer may recur again. Thus, to completely cure cancer, it is important to study the cancer stem cell.

Furthermore, to confirm the cancer stem cell, a cancer stem cell marker may be used. The cancer stem cell marker may be CD47, BMI-1, CD24, CXCR4, DLD4, GLI-1, GLI-2, PTEN, CD166, ABCG2, CD171, CD34, CD96, TIM-3, CD38, STRO-1 and CD19, and specifically, it may be CD44, CD133, ALDH1A1, ALDH1A2, EpCAM, CD90 and LGR5, but not limited thereto.

The method of preparing and kit for preparing cancer stem cell spheroids of the present invention have an advantage capable of preparing cancer stem cells more simply and rapidly, since artificial gene manipulation is not required for preparing a spheroid.

In addition, it has been confirmed that a cancer stem cell (CSC) marker gene prepared by the method and kit is expressed (Example 6), and has a drug resistance property by drug discharging, and has a cancer-formation ability in vivo (Example 12), and therefore the cancer stem cell spheroid prepared by the method and kit of the present invention may be used for studying cancer stem cells and screening its therapeutic agent by having properties of the caner stem cell.

The cancer stem cell spheroids of the present invention may be cultured in a three-dimensional, stereoscopic culture form, and may be cancer stem cell spheroids which have a characteristic of drug resistance or is cancer cell-derived patient-specific, but not limited thereto.

The term of the present invention, “albumin” consists of basic substances of cells with globulin, and it is comprised in the culture medium of cancer cells plated in the cell culture substrate of the present invention, and substances capable of forming cancer stem cell spheroids from cancer cells are included without limitation. The albumin of the present invention may be selected from the group consisting of serum albumin, ovalbumin, lactalbumin and combinations thereof, but not limited thereto. As the example, a commercially available serum replacement (SR) is also included, but not limited thereto. Most of cells require serum to proliferate, and artificial serum or serum replacement which can perform an equal or similar function to natural serum may be used. The artificial serum or serum replacement may be used as a substitute for natural serum in cell culture, and it commonly comprises albumin. The albumin of the present invention may be added as a single component of albumin, or be provided as a formulation comprised in a serum replacement, a formulation prepared by adding the albumin additionally to a serum replacement, or a formulation prepared by adding the albumin additionally to FBS, and more preferably, it may be provided as a formulation in which albumin is further added to a serum replacement, but not limited thereto. In addition, the serum albumin may be selected from the group consisting of bovine serum albumin, human serum albumin and combinations thereof depending on its origin, but not limited thereto. Herein, it has been confirmed that the spheroid prepared using bovine serum albumin expresses a cancer stem cell-related marker (Example 6), and therefore it can be seen that albumin can induce a cancer stem cell.

The albumin concentration may be comprised in a medium at a concentration of 0.1 mg/ml to 500 mg/ml. Specifically, the albumin concentration may be comprised in a medium at a concentration of about 0.1, 0.2, 0.5, 0.6, 1, 1.1, 2, 3, 4, 5, 6, 11, 16, 21, 26, 31, 36, 41, 46, 51, 56, 61, 66, 71, 76, 81, 86, 91, 96, 100, 101, 106, 111, 116, 121, 126, 131, 136, 141, 146 mg/ml or more, or about 500, 450, 400, 350, 300, 250, 200, 199, 195, 190, 175, 170, 150, 149, 144, 139, 134, 129, 124, 119, 114, 109, 104, 99, 94, 89, 84, 79, 74, 69, 64, 59, 54, 49, 44, 39, 34, 29, 24, 19, 14, 9, 4, 1.4, 0.9, 0.4 mg/ml or less, more specifically, about 0.1 mg/ml to about 500 mg/ml, about 0.5 mg/ml to about 500 mg/ml, about 1 mg/ml to about 500 mg/ml, about 5 mg/ml to about 500 mg/ml, about 10 mg/ml to about 500 mg/ml, about 20 mg/ml to about 500 mg/ml, about 40 mg/ml to about 500 mg/ml, about 0.1 mg/ml to about 200 mg/ml, about 0.5 mg/ml to about 200 mg/ml, about 1 mg/ml to about 200 mg/ml, about 5 mg/ml to about 200 mg/ml, about 10 mg/ml to about 200 mg/ml, about 20 mg/ml to about 200 mg/ml, about 40 mg/ml to about 200 mg/ml, about 0.1 mg/ml to about 150 mg/ml, about 0.5 mg/ml to about 150 mg/ml, about 1 mg/ml to about 150 mg/ml, about 5 mg/ml to about 150 mg/ml, about 10 mg/ml to about 150 mg/ml, about 20 mg/ml to about 150 mg/ml, about 40 mg/ml to about 150 mg/ml, about 0.1 mg/ml to about 100 mg/ml, about 0.5 mg/ml to about 100 mg/ml, about 1 mg/ml to about 100 mg/ml, about 5 mg/ml to about 100 mg/ml, about 10 mg/ml to about 100 mg/ml, about 20 mg/ml to about 100 mg/ml, about 40 mg/ml to about 100 mg/ml, about 0.1 mg/ml to about 80 mg/ml, about 0.5 mg/ml to about 80 mg/ml, about 1 mg/ml to about 80 mg/ml, about 5 mg/ml to about 80 mg/ml, about 10 mg/ml to about 80 mg/ml, about 20 mg/ml to about 80 mg/ml, about 40 mg/ml to about 80 mg/ml, about 0.1 mg/ml to about 70 mg/ml, about 0.5 mg/ml to about 70 mg/ml, about 1 mg/ml to about 70 mg/ml, about 5 mg/ml to about 70 mg/ml, about 10 mg/ml to about 70 mg/ml, about 20 mg/ml to about 70 mg/ml, about 40 mg/ml to about 70 mg/ml, about 0.1 mg/ml to about 60 mg/ml, about 0.5 mg/ml to about 60 mg/ml, about 1 mg/ml to about 60 mg/ml, about 5 mg/ml to about 60 mg/ml, about 10 mg/ml to about 60 mg/ml, about 20 mg/ml to about 60 mg/ml, about 40 mg/ml to about 60 mg/ml, about 0.1 mg/ml to about 50 mg/ml, about 0.5 mg/ml to about 50 mg/ml, about 1 mg/ml to about 50 mg/ml, about 5 mg/ml to about 50 mg/ml, about 10 mg/ml to about 50 mg/ml, about 20 mg/ml to about 50 mg/ml, about 40 mg/ml to about 50 mg/ml, about 0.1 mg/ml to about 40 mg/ml, about 0.5 mg/ml to about 40 mg/ml, about 1 mg/ml to about 40 mg/ml, about 5 mg/ml to about 40 mg/ml, about 10 mg/ml to about 40 mg/ml, about 20 mg/ml to about 40 mg/ml, or about 40 mg/ml, and may be comprised in a medium at a concentration of albumin comprised in a serum replacement, but not limited thereto. More preferably, the albumin concentration may be comprised in a medium at a concentration of 0.1 mg/ml to 400 mg/ml, or 0.1 mg/ml to 200 mg/ml. Further preferably, the albumin concentration may be comprised in a medium at a concentration of 0.5 mg/ml to 400 mg/ml, 0.5 mg/ml to 200 mg/ml, or 0.5 mg/ml to 100 mg/ml.

Herein, the term, “about” includes all of ±0.5, ±0.4, ±0.3, ±0.2, ±0.1, and the like, and about includes all the numerical values equal or similar to the numerical value behind the term, but not limited thereto.

Herein, the term, “culture” means growing a cell under a suitably controlled environment condition, and the culture process of the present invention may be conducted according to suitable medium and culture conditions known in the art. This culture process may be adjusted and used by those skilled in the art according to the selected cell. Specifically, herein, to prepare cancer stem cell spheroids, it may be cultured in an albumin-containing medium, and as the example, it may be cultured in a serum replacement (SR)-containing medium, but not limited thereto.

Other aspect of the present invention provides cancer stem cell spheroids prepared by the method of preparing. The “cancer stem cell” and “spheroid” are as described above.

Other aspect of the present invention provides a kit for preparing cancer stem cell spheroids, comprising a composition for inducing cancer stem cells from cancer cells comprising a medium for cell culture containing a cell culture substrate and albumin, wherein the cell culture substrate comprises a cyclosiloxane polymer and the medium comprises albumin.

The “cell culture substrate comprising a cyclosiloxane polymer”, “albumin”, “cancer stem cell” and “spheroid” are as described above.

The kit of the present invention can prepare a caner stem cell spheroid. The kit may comprise a cell culture substrate and a medium as basic composition, and specifically, the cell culture substrate may be a substrate comprising a polymer formed by a cyclosiloxane compound, but any substrate which can prepare or culture cancer stem cell spheroids is included without limitation. In addition, the medium may be specifically an albumin-containing medium or serum replacement-containing medium, but any medium which can prepare or culture cancer stem cell spheroids is included without limitation. In the kit, instructions for the method for preparing cancer stem cell spheroids may be further comprised.

Other aspect of the present invention provides a method for screening a drug for treating cancer cell resistance, comprising (a) preparing cancer stem cell spheroids by the method of preparing; (b) treating a candidate substance for treating cancer cell resistance to the cancer stem cell spheroid of the (a) step; and (c) comparing cancer stem cell spheroids group of the (b) step in which the candidate substance for treating cancer cell resistance is treated and a control group in which the candidate substance for treating cancer cell resistance is not treated. The “cancer stem cell” and “spheroid” are as described above.

The comparing cancer stem cell spheroids group in which the candidate substance for treating cancer cell resistance is treated and a control group in which the candidate substance for treating cancer cell resistance is not treated of the (c) step may comprise measuring and computing the expression level of cancer stem cell markers, and the measuring the expression level of cancer stem cell markers may use common methods for measuring the expression level used in the art without limitation, and as the example, there is western blot, ELISA, radioimmunoassay, radioimmunodiffusion, Ouchterlony immunodiffusion, Rocket immunoelectrophoresis, immunohistostaining, immunoprecipitation assay, complement fixation assay, FACS or protein chip method, or the like.

The term of the present invention, “candidate substance” is a substance expected to treat cancer or substance expected to improve its prognosis, and specifically, may be a substance capable of treating cancer or improving prognosis by removing cancer stem cells and inhibiting cancer cell resistance, and any substance expected to directly or indirectly enhancing or improving cancer or a cancer stem cell is included without limitation. The example of the candidate substance includes all predicted therapeutic substances such as compounds, genes or proteins, or the like. The screening method of the present invention may confirm the expression level of cancer stem cell markers before and after administration of the candidate substance, and also, determine the corresponding candidate substance as a predicted therapeutic agent for cancer stem cells or cancer cell resistance, when the expression level is reduced compared to that before administering the candidate substance.

In addition, the (b) step may further comprise treating with a drug having resistance, but not limited thereto.

### Advantageous Effects

The method of producing and kit for producing cancer stem cell spheroids of the present invention can conveniently produce cancer stem cell spheroids, and the cancer stem cell spheroid prepared by the method and kit can be effectively utilized for screening drugs for treating cancer cell resistance.

### BRIEF DESCRIPTION OF THE DRAWINGS

FIG. 1a to FIG. 1f show structures of the compounds used for PTF manufacture, and FIG. 1g to FIG. 1l show structures of various cyclosiloxane compounds, and FIG. 1m is a drawings which shows the process of forming a spheroid having cancer-formation ability on a specific PTF surface, and FIG. 1n is a drawing which confirms the formation of a spheroid having cancer-formation ability on various functional PTFs, and FIG. 1o to 1t are drawings which show that a spheroid is formed on a substrate comprising various cyclosiloxane compounds.

FIG. 2a is a drawing which confirms whether various human cancer cell lines form a spheroid on a surface of pV4D4 PTF, and FIG. 2b is a drawing which confirms whether various human cancer cell lines form any type of spheroid on a surface of pV4D4 PTF.

FIG. 3a is a drawing which shows an FT-IR spectrum of V4D4 monomer and pV4D4 PTF, and FIG. 3b is a drawing which shows the result of XPS survey scan of pV4D4 PTF, and FIG. 3c is a drawing which shows the water contact angles of the uncoated Si wafer, pV4D4-coated Si wafer, uncoated cell culture substrate, and pV4D4-coated cell culture substrate, and FIG. 3d is a drawing which shows the AFM images of uncoated TCP and pV4D4-coated TCP.

FIG. 4 is a drawing which confirms the formation of a spheroid on pV4D4-coated TCP having a PTF thickness of 10, 50, 100, 200, and 300 nm.

FIG. 5a is a drawing which shows the expression level of CD133 and CD44 of cells cultured in various kinds of media containing FBS and SR, and FIG. 5b is a drawing which confirms the albumin content of FBS and SR by western blot.

FIG. 6a is an image which shows spheroid formation according to the concentration of BSA comprised in a serum-free medium (SFM), and FIG. 6b is a drawing which shows the expression level of CD133 according to the concentration of BSA.

FIG. 7a is a drawing which shows the CD133 expression level of the cell cultured in a serum-free medium (SFM) containing FBS, SR or BSA of 40 mg/ml in TCP or pV4D4.

FIG. 7b is a drawing which shows the spheroid formation of three kinds of cancer cells cultured in a BSA-containing serum-free medium (SFM) in pV4D4.

FIG. 7c is a graph which shows the expression level of CD133 that is a cancer stem cell marker gene of the spheroid produced in a substrate comprising various cyclosiloxane compounds, and in the x axis of FIG. 7c, 1g shows the CD133 expression of the cancer stem cell spheroid produced in a substrate in which pV4D4, and cyclosiloxane compounds of FIG. 1g are copolymerized, and 1h shows the CD133 expression of the cancer stem cell spheroid produced in a substrate in which pV4D4, and cyclosiloxane compounds of FIG. 1h are copolymerized, and 1i shows the CD133 expression of the cancer stem cells spheroid produced in a substrate in which pV4D4, and cyclosiloxane compounds of FIG. 1i are copolymerized, and 1j shows the CD133 expression of the cancer stem cells spheroid produced in a substrate in which pV4D4, and cyclosiloxane compounds of FIG. 1j are copolymerized, and 1k shows the CD133 expression of the cancer stem cells spheroid produced in a substrate in which pV4D4, and cyclosiloxane compounds of FIG. 1k are copolymerized, and 1l shows the CD133 expression of the cancer stem cells spheroid produced in a substrate in which pV4D4, and cyclosiloxane compounds of FIG. 1l are copolymerized.

FIG. 7d is a drawing which shows measuring the CD133 expression level after culturing SKOV3 in a substrate comprising a cyclosiloxane polymer according to various albumin concentrations.

FIG. 7e is a graph showing the expression level of CD133 of the spheroid formed by culturing cancer cells in a BSA-added medium so that the concentration of albumin is 0, 0.01 mg/ml, 0.1 mg/ml, 1 mg/ml, 10 mg/ml, 100 mg/ml, 200 mg/ml, and 400 mg/ml in SFM medium, in a substrate comprising a cyclosiloxane compound, according to the concentration of albumin.

FIG. 8a is a drawing which shows the shapes of the SKOV3 spheroids produced using hanging-drop, U-bottom, ULA and pV4D4.

FIG. 8b is a drawing which shows the laminin expression pattern in the SKOV3 spheroids produced on the ULA or pV4D4 surface, and red represents laminin and blue represents nuclei.

FIG. 8c is a drawing which shows the ALDH1A1 mRNA expression level of the SKOV3 spheroids produced using hanging-drop, U-bottom, ULA and pV4D4.

FIG. 8d is a drawing which shows the Oct3/4, Sox2 and Nanog mRNA expression level in SKOV3-ssiCSCs (4 days and 8 days) on the pV4D4 surface.

FIG. 9 is a drawing which shows the result of the wound healing assay (a) and invasion assay (b) of SKOV3-ssiCSCs produced on the pV4D4 surface.

FIG. 10 is a drawing which confirms the spheroid formation by SKOV3-ssiCSCs and U87MG-ssiCSCs.

FIG. 11 is a drawing which shows the CSC-related marker mRNA expression level (a and b) and the flow cytometry result (c), in SKOV3-, MCF-7-, Hep3B and SW480-ssiCSC spheroids cultured on the pV4D4 surface for 4 days and 8 days.

a and b of FIG. 12 are drawings which show the side-population assay result (a) and the cell viability for doxorubicin (b), of SKOV3-ssiCSC, MCF-7-ssiCSC, Hep3B-ssiCSC and SW480-ssiCSC spheroids cultured on the pV4D4 surface for 4 days and 8 days, and c is a drawing which shows the cell viability for doxorubicin in a cell in which SW480-ssiCSCs are subcultured once or twice, and d is a drawing which shows the mRNA expression level of the drug discharge ABC transporter-related gene of SKOV3-ssiCSCs produced by culturing for 8 days.

a of FIG. 13 is a drawing which shows the process of forming tumor by administering SKOV3-ssiCSC spheroid-derived cells to a BABL/c nude mouse, and b is a drawing which shows the tumor-metastasized liver, and c is a drawing of H&E staining the tumor-metastasized liver and observing it, and d is a drawing which shows lesions metastasized in the liver of the BABL/c nude mouse in which the SKOV3-ssiCSC spheroid-derived cell is injected, and e is a drawing of staining TNC to the tumor-metastasized liver and observing it.

a of FIG. 14 shows the heat map of Wnt target gene of the SKOV3-ssiCSC spheroid (n=46), and b shows the expression (1 day, 4 days and 8 days) of DKK1 in SKOV3-ssiCSCs and the expression (4 days and 8 days) level of AXIN2 and MMP-2 mRNA in SKOV3-ssiCSCs, and c shows the western blot result of phosphorylated 0-catenin and the entire 0-catenin of SKOV3-ssiCSCs (4 days and 8 days), and d is a drawing which shows the location of 0-catenin in cells of SKOV3-ssiCSCs, and e is a drawing which shows the TNC expression in SKOV3-ssiCSCs.

FIG. 15 is a drawing which shows the TNC expression (a) and DKK1 mRNA expression level (b) in MCF-7-ssiCSC, Hep3B-ssiCSC, and SW480-ssiCSC spheroids.

FIG. 16a is a drawing which shows observing the spheroid formed by culturing cancer cells in a BSA-added FBS medium, on a substrate comprising a cyclosiloxane compound, with a microscope.

FIG. 16b is a graph showing the DKK-1 gene expression level of the spheroid formed by culturing cancer cells in a BSA-added FBS medium, on a substrate comprising a cyclosiloxane compound, based on Beta-actin (housekeeping gene).

FIG. 16c is a graph showing the DKK-1 gene expression level of the spheroid formed by culturing cancer cells in a BSA-added FBS medium, on a substrate comprising a cyclosiloxane compound, based on GAPDH (housekeeping gene).

### DETAILED DESCRIPTION OF THE EMBODIMENTS

Hereinafter, the present invention will be described in more detail by referential examples, comparative examples and examples. However, these referential examples, comparative examples and example are intended to exemplarily illustrate the present invention, but the scope of the present invention is not limited to these referential examples, comparative examples and examples.

### Referential Example 1: Heterologous Tumor Formation Analysis

Female BALB/c nude mice (6 weeks) were obtained from Orient Bio Inc., and were stored in an aseptic condition in the animal laboratory of Korea Advanced Institute of Science and Technology. The mice were randomly assigned in random experimental groups. All operations were performed under isoflurane anesthesia, and for ethical procedures and scientific management, all the animal-related procedures were examined and approved by Korea Advanced Institute of Science and Technology, Institutional Animal Care and Use Committee (KAIST-IACUC) (Approval number: KA2014-21).

In addition, to prepare a human ovarian cancer heterologous model, different series of concentrations (10⁶ to 10² cells) of 2D-cultured control SKOV3 cell or SKOV3-ssiCSC isolated from a spheroid corresponding thereto was mixed with 50% Matrigel (Corning), and then was subcutaneously injected to 6-week female BALB/c nude mice. Tumor formation was monitored for 130 days at maximum, and it was recorded that tumor was formed when the tumor volume reached about 50 mm³. To prepare a human breast cancer heterologous model, different series of concentrations (10⁷ to 10² cells) of 2D control cell or ssiCSC derived from MCF7-Luc cancer cell was subcutaneously injected to 6-week female BALB/c nude mice. 50 μl sesame oil (Sigma) dissolved in 0-estradiol 17-valerate (2.5 μg; Sigma) was subcutaneously administered to BALB/c nude mice through a neck every 10 days. To prepare a human glioma heterologous model, different series of concentrations (10⁶ to 10² cells) of 2D control U87MG cell, ULA-cultured U87MG spheroid or pV4D4-cultured U87MG-ssiCSC cell was mixed with 50% Matrigel, and was subcutaneously injected to 6-week female BALB/c nude mice. Tumor formation from MCF7-Luc and U87MG was monitored by 90 days, and it was recorded that tumor was formed when the tumor volume reached about 50 mm³.

### Referential Example 2: Cell Viability Analysis

ssiCSC spheroids prepared from different kinds of cancer cells (SKOV3, MCF-7, Hep3B and SW480) were isolated using trypsin (TrypLE Express, Gibco), and the isolated cells were washed with D-PBS twice. The ssiCSC was plated on a 96-well plate (1×10⁴ cells/well) and was cultured in a cell growth medium at 37° C. for 24 hours. Then, the medium was removed, and a new medium comprising various concentrations of doxorubicin was added to each well and cultured for 24 hours. Next, each well was washed with D-PBS and was replaced with a new cell growth medium of 100 μl, and then WST-1 cell proliferation reagent (Roche) of 10 μl was added and cultured for 4 hours. Then, the absorbance at 450 nm (standard wavelength, 600 nm) was measured using a microplate reader (Molecular Devices).

### Referential Example 3: Histological Analysis and Immunohistochemistry

Liver biopsy samples obtained from BALB/C nude mice inoculated by the 2D control group or SKOV3-ssiCSC cancer cell were fixed with 10% formalin, dehydrated and embedded with paraffin, and cut into samples in a thickness of 5 μm, and placed on a slide. The samples were dewaxed and stained with hematoxylin % eosin (H&E) for histological evaluation with a standard optical microscope (Eclipse 80i, Nickon).

Liver metastasis was confirmed by an immunohistochemical method after embedding tissue with paraffin and fragmentating it (5 μm). The fragmented liver tissue was sterilized with 10 mM sodium citrate buffer (pH 6.0) for antigen recovery, and blocked with PBS containing 5% bovine serum albumin (BSA) and 1% goat serum, and then incubated with a rabbit anti-human TNC primary antibody at a room temperature (RT) for 1 hour (20 μg/ml; cat. no. AB19011; Millipore). After incubation, the slide was washed with D-PBS, and incubated with a biotin-attached anti-rabbit secondary antibody (1:200; Vector Laboratories) at a room temperature for 30 minutes, and then incubated with HRP (horseradish peroxidase, 1:500, Vector) at a room temperature for 30 minutes. The immunoreactive protein was visualized using a substrate, 3,3-diaminobenzidine (Vector Laboratories), and then counterstained using hematoxylin.

### Referential Example 4: Western Blot Analysis

2D control SKOV3 cells and SKOV3-ssiCSC spheroids were dissolved with RIPA dissolution buffer containing proteinase inhibition cocktail (ThermoFisher Scientific) on ice for 30 minutes. Using Bradford protein analysis kit (Bio-Rad), the protein of the lysates was quantified, and the equivalent amount of protein (50 μg) was isolated by electrophoresis using Bolt 4-12% Bis-Tris Plus polyacryl amide gel (ThermoFisher Scientific). According to the manufacturer's instructions, the gel was dry blotted on a PVDF (polyvinylidene difluoride) film using iBlot2 transfer system (ThermoFisher Scientific).

The PVDF film was immunoblotted by incubating with a primary rabbit anti-phospho-β-catenin antibody (1:1000, cat. no. 9561; Cell Signaling Technology), a mouse anti-β-catenin antibody (1:1000, cat. no. 13-8400; Invitrogen), and a rabbit anti-GAPDH antibody (1:1000, cat. no. 25778; Santa Cruz Biotechnology), and then using standard procedures, it was incubated suitably with an HRP-bound anti-rabbit IgG secondary antibody (1:5000, cat. no. 31460; Invitrogen) or an anti-mouse IgG (1:5000, cat. no. 31430; Invitrogen) secondary antibody. The protein was visualized using SuperSignal West Pico Chemiluminescent Substrate (ThermoFisher Scientific) and ChemiDoc MP system (Bio-Rad).

### Referential Example 5: Flow Cytometry

Flow cytometry was performed as follows. Specifically, after treating 2D control cancer cells and ssiCSC spheroids corresponding thereto, which were cultured as a single layer (cultured for 8 days) with trypsin, the cells were isolated with buffer [D-PBS containing 1% FBS (fetal bovine serum)], respectively. SKOV3, MCF-7, Hep3B, and SW480 cancer cells were stained with an APC (allophycocyanin)-conjugated anti-CD133 primary antibody (1:100; eBioScience), an FITC-conjugated anti-CD44 primary antibody (1:200; BD Biosciences), an PE (phycoerythrin)-conjugated anti-CD90 primary antibody (1:100, MACS; Miltenyi Biotec), and an FITC-conjugated anti-CD133 primary antibody (1:100; Miltenyi Biotec), and were analyzed using a flow cytometry system (BD Calibur and BD LSR Fortessa).

In addition, for side population assays, 2D control cancer cells and ssiCSCs were isolated using trypsin, and stained with Hoechst 33342 (ThermoFisher Scientific) in DMEM containing 2% FBS and 10 mM HEPES buffer at 37° C. for 90 minutes. Then, the cells were washed with HBSS containing 2% FBS and analyzed using a flow cytometry system (BD LSR Fortessa). The flow cytometry data histogram and plot were analyzed using FlowJo software (Tree Star Inc.).

### Referential Example 6: Live Cell Imaging

ssiCSC spheroids were imaged using LumaScope 620 system (Etaluma) allowing live ell imaging in a standard incubator (humidification 5% carbon dioxide, 37° C.). Phase difference images were observed using a 10× object lens every 2.5 minutes for 24 hours.

### Referential Example 7: RNA Extraction and mRNA Sequencing

According to the manufacturer's protocol, mRNA was extracted from SKOV3 spheroids and 2D control SKOV3 cells which were cultured on an pV4D4-coated plate for 8 days, using a magnetic mRNA separation kit (NEB). As described in the manufacturer's protocol, using DNase-treated mRNA and NEXTflex Rapid Directional mRNA-Seq kit (BIOO), libraries were manufactured. Each library was sequenced using a single-end method (50-bp reads) in HiSeq2500 system. The sequenced result was compared with human genome (Hg19 version) using STAR aligner (v.2.4.0) 61.

In addition, to investigate DEG, HOMER software algorithm and DESeq R package were used. Heatmap and MA plot were visualized using pheatmap function and plotMA function of R statistical programming language v.3.3.0 (http://www.r-project.org/), respectively.

### Referential Example 8: Immune Staining Method for Immunocytochemistry

SKOV3 spheroids were transferred from ULA plate and pV4D4 plate to a 1.5-ml tube, and incubated in 4% paraformaldehyde solution (Sigma) at a room temperature for 30 minutes to fix the spheroids. The fixed spheroids were incubated in D-PBS (Dulbecco's phosphate-buffered saline) solution containing 0.25% (w/v) Triton X-100 (Sigma) at a room temperature for 10 minutes, and washed with D-PBS, and then for blocking, incubated with D-PBS containing 3% BSA.

To staining the spheroids with laminin, the fixed spheroids were incubated with an anti-human laminin primary rabbit antibody (1:100, cat. no. 11575; Abcam) at 4° C. for 12 hours. Then, after washing with D-PBS, obtained spheroids were incubated with a rhodamine red-X-conjugated anti-rabbit secondary antibody (1:500, cat. no. R6394; Invitrogen) at a room temperature for 1 hour, and then incubated with Hoechst 33342 for 10 minutes.

In addition, for TNC staining, SKOV3 2D control group or SKOV3 spheroids were incubated with an anti-human TNC primary rabbit antibody (20 μg/ml, cat. no. AB19011; Millipore) at 4° C. for 12 hours. Then, after washing with D-PBS, the cells and spheroids were incubated with an FITC-conjugated anti-rabbit secondary antibody (1:500, cat. no. sc-2012; Santa Cruz) at a room temperature for 1 hour. Then, they were incubated with Hoechst 33342 for 10 minutes.

For β-catenin staining, SKOV3 2D control group and SKOV3-ssiCSCs were incubated with a mouse anti-human β-catenin primary antibody (1:100, cat. no. 13-8400; Invitrogen) at a room temperature for 1 hour. Then, after washing with D-PBS, the cells were incubated with a TRITC-conjugated anti-mouse secondary antibody (1:1000, cat. no. ab6786; Abcam) at a room temperature for 1 hour, and then incubated with Hoechst 33342 for 10 minutes. All fluorescent images were visualized using a confocal laser-scanning microscope (LSM 780, Carl Zeiss).

### Referential Example 9: Statistical Analysis and Data Reference

Data were represented by mean±standard deviation (s.d.). Using unpaired Student's t-test of GraphPad Prism software (La Jolla), statistical analysis was performed. P value<0.05 was considered as statistically significant.

In addition, GSE106848 RNA sequencing data of Gene Expression Omnibus data storage of NCBI were used.

### Example 1: Production of Cell Culture Substrate or Cover Glass Comprising Cyclosiloxane Polymer

1-1: Production of PTF Cell Culture Substrate or Cover Glass Through iCVD Process

A polymer thin film (PTF) comprising a polymer formed by a cyclosiloxane compound was prepared by the following method.

At first, pV4D4 [poly(2,4,6,8-tetravinyl-2,4,6,8-tetramethyl cyclotetrasiloxane) polymer thin film (PTF) was prepared. Specifically, for evaporation of monomers, V4D4 [2,4,6,8-tetravinyl-2,4,6,8-tetramethyl cyclotetrasiloxane] (99%; Gelest) and tert-butyl peroxide (TBPO, 98%; Aldrich) were heated to 70 and 30, respectively. The evaporated V4D4 and TBPO were introduced into iCVD chamber (Daeki Hi-Tech Co. Ltd.) at a flow rate of 1.5 and 1 standard cm³/min (sccm). The substrate temperature was maintained at 40, and the filament temperature was maintained at 200, and the pressure of the iCVD chamber was set to 180 mTorr. The deposition rate of pV4D4 film was estimated to be 1.8 nm/min. The thickness of the pV4D4 film was monitored at the position using an He—Ne laser (JDS Uniphase) interferometer system.

1-2: Production of Cell Culture Substrate Comprising Various Cyclosiloxane Polymers

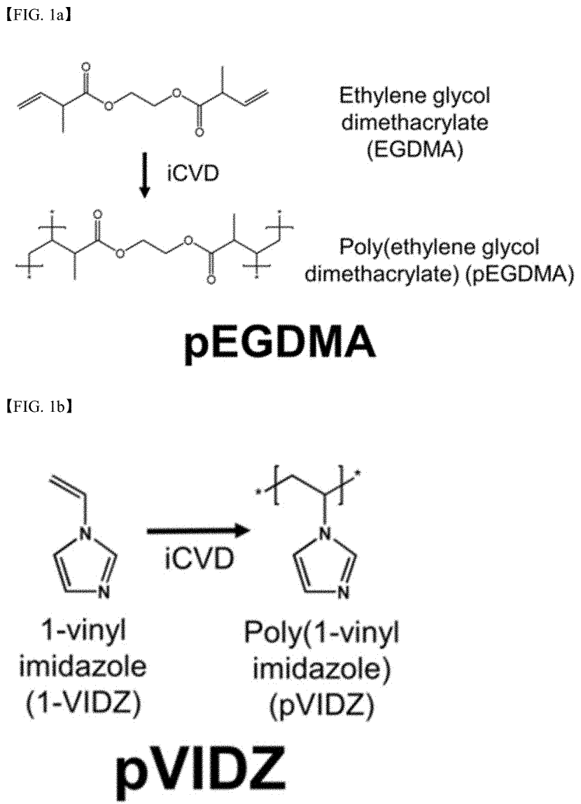

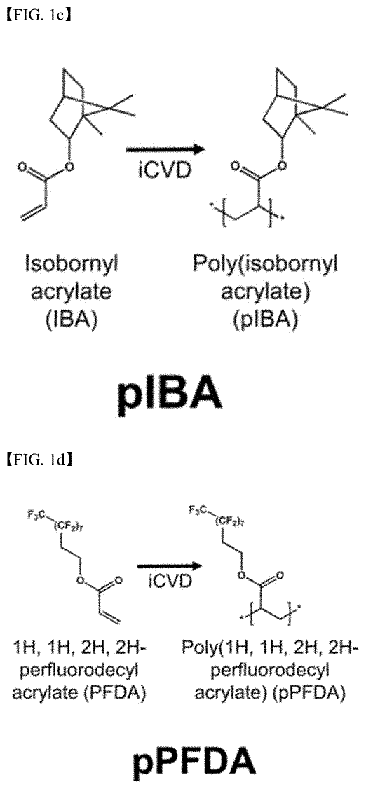

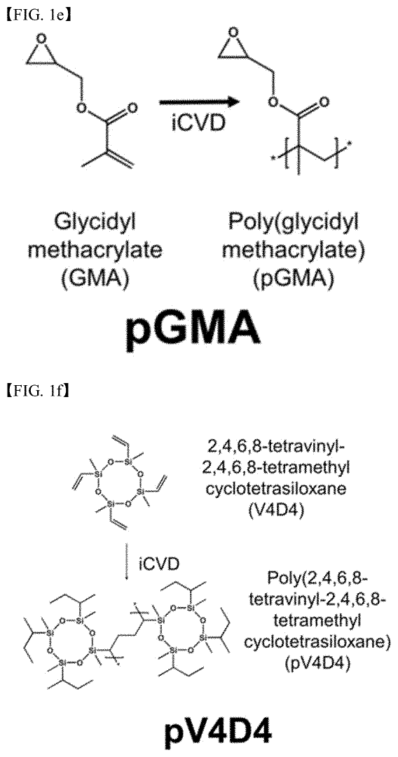

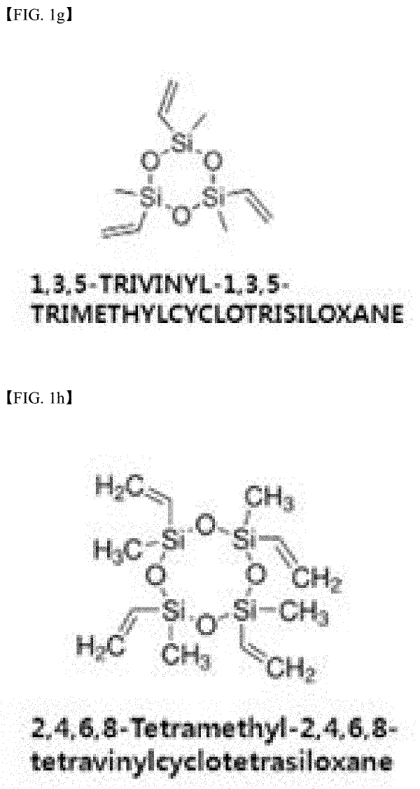

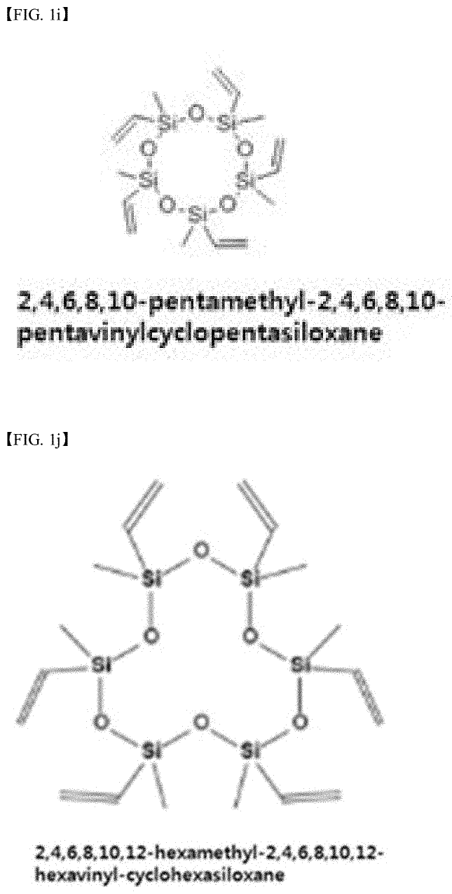

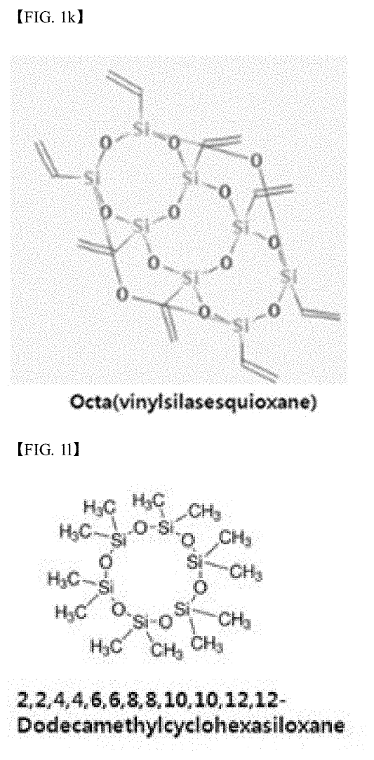

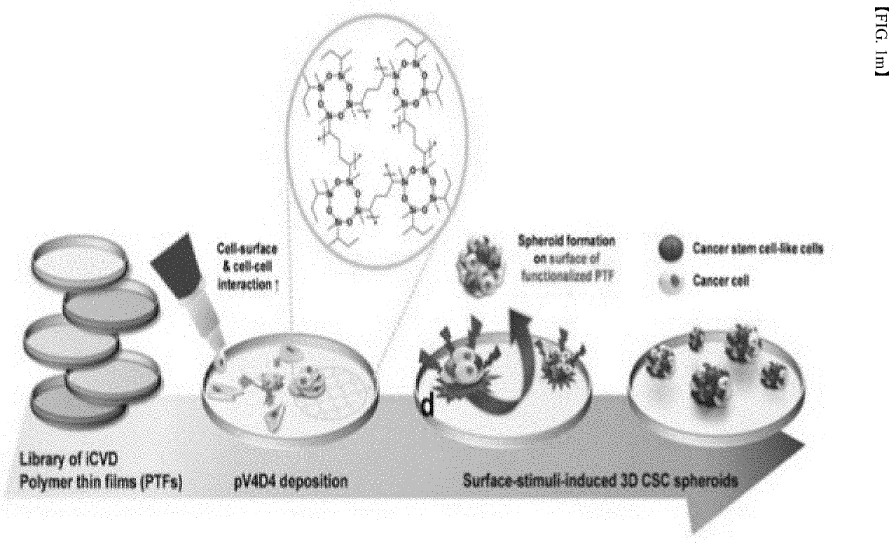

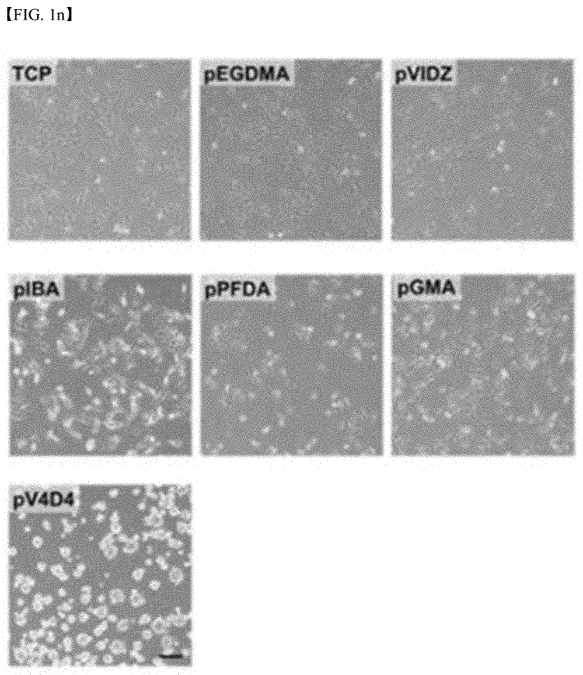

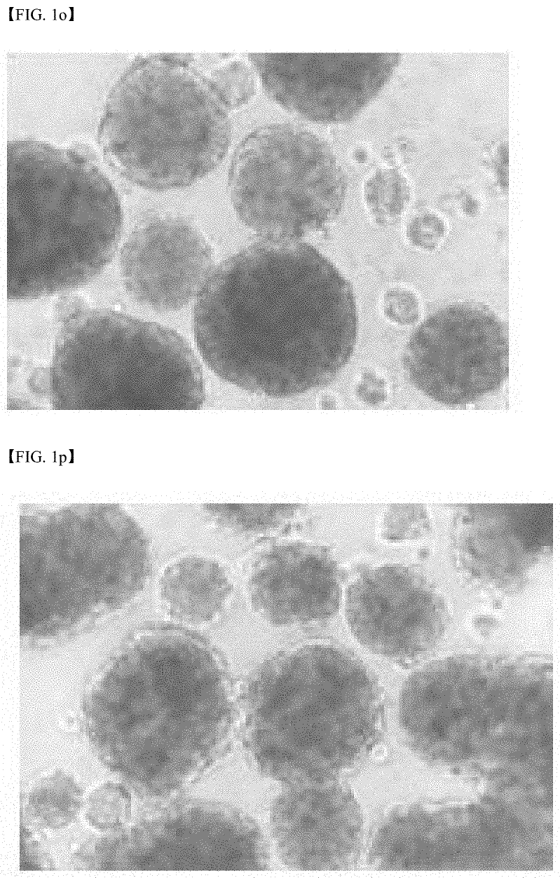

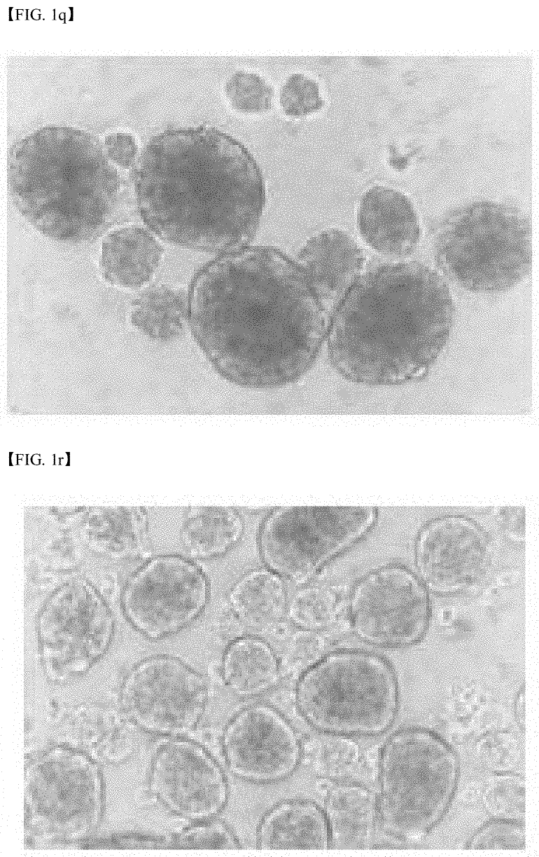

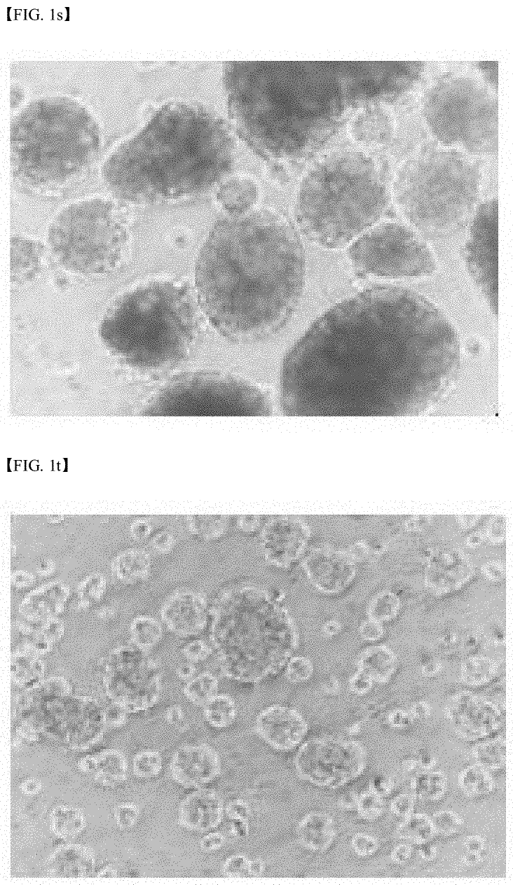

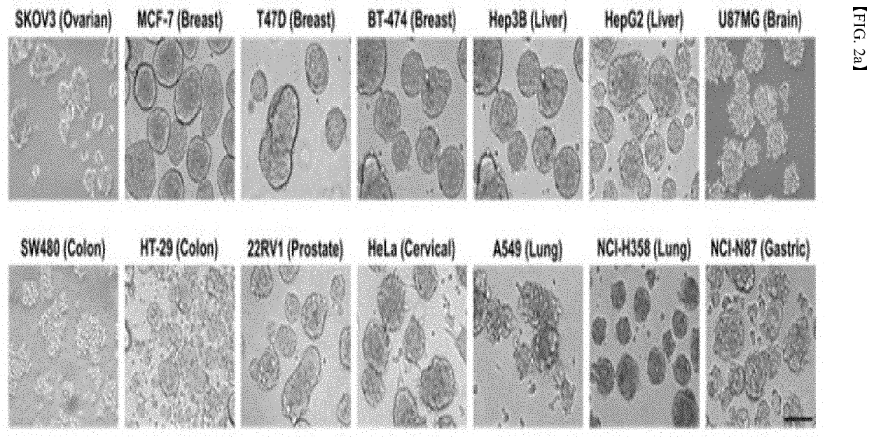

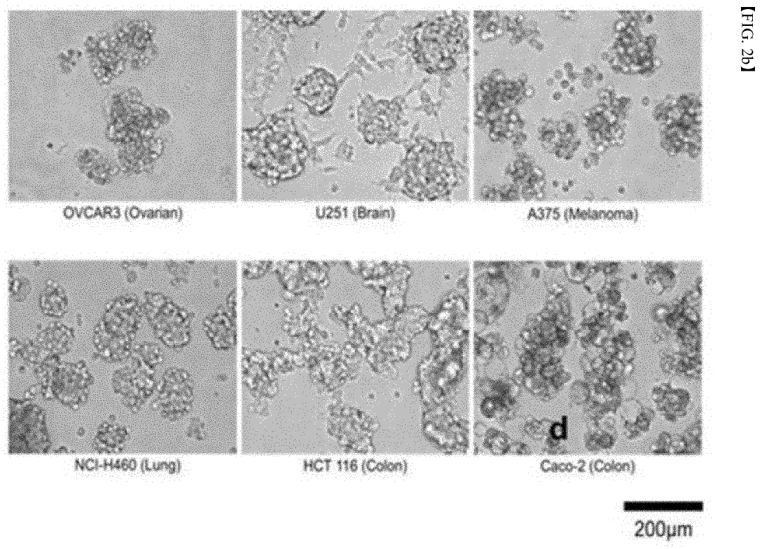

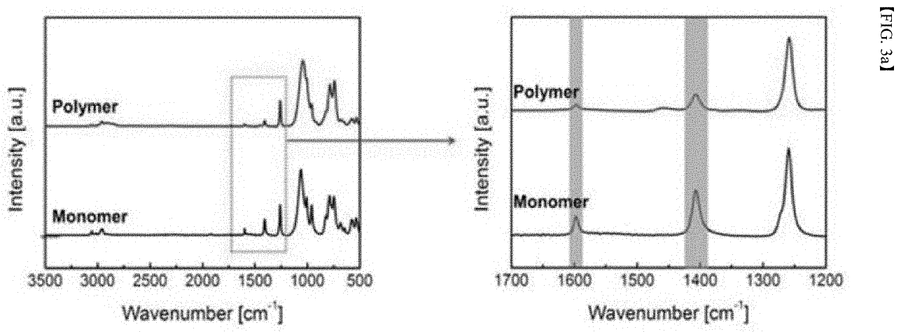

To produce cell culture substrates comprising various cyclosiloxane compounds, using 1,3,5-trivinyl-1,3,5-trimethylcyclotrisiloxane, 2,4,6,8-tetramethyl-2,4,6,8-tetravinylcyclotetrasiloxane (V4D4), 2,4,6,8,10-pentamethyl-2,4,6,8,10-pentavinylcyclopentasoxane, 2,4,6,8,10,12-hexamethyl-2,4,6,8,10,12-hexavinyl-cyclohexasiloxane, octa(vinylsilasesquioxane), and 2,2,4,4,6,6,8,8,10,10,12,12-dodecamethylcyclohexasiloxane, copolymer substrates were formed at a ratio of 1:9 with pV4D4, respectively. The chemical structures of the various cyclosiloxane compounds were shown in FIG. 1g to FIG. 1l.

FIG. 1g to FIG. 1l shows the structures of the various cyclosiloxane compounds, and FIG. 1g shows the structure of 1,3,5-trivinyl-1,3,5-trimethylcyclotrisiloxane, and FIG. 1h shows the structure of 2,4,6,8-tetrametyl-2,4,6,8-tetravinylcyclotetrasiloxane (V4D4), and FIG. 1i shows the structure of 2,4,6,8,10-pentamethyl-2,4,6,8,10-pentavinylcyclopentasiloxane, and FIG. 1j shows the structure of 2,4,6,8,10,12-hexamethyl-2,4,6,8,10,12-hexavinyl-cyclohexasiloxane, and FIG. 1k shows the structure of octa(vinylsilasesquioxane), and FIG. 1l shows the structure of 2,2,4,4,6,6,8,8,10,10,12,12-dodecamethylcyclohexasiloxane.

1-3: Analysis Method

Fourier-transform infrared spectrums (FT-IR) of the V4D4 monomer and pV4D4 polymer were obtained using 64 mean scans and 0.085 cm⁻¹ optical resolution in a normal absorbance mode using ALPHA FTIR spectrometer (Bruker Optics, USA). Each spectrum was calibrated at baseline and recorded in the 400-4000 cm⁻¹ range.

The chemical composition of the pV4D4 PTF surface was analyzed by X-ray photoelectron spectroscopy (XPS; K-alpha, Thermo VG Scientific Inc.) under the atmospheric pressure of 2.0×10⁻⁹ mbar. The XPS spectrum was recorded in the 100-1100 eV range using a monochromatic Al Kα radiation X-ray source with kinetic energy (KE) of 12 kV and 1486.6 eV.

The surface topography in the 45×45 μm region was analyzed by an atomic force microscope (AFM; PSIA XE-100, Park Systems) at a scan rate of 0.5 Hz in a non-contact mode.

The water contact angles for the Si wafer, pV4D4-coated Si wafer, tissue culture substrate and pV4D4-coated substrate were measured using a contact angle analyzer (Phoenix 150; Surface Electro Optics, Inc.) by dropping 10 μl deionized water on the corresponding surface.

### Example 2: Formation of Cancer Cell-Derived Spheroids Using Various Polymer Thin Films (PTF)

2-1: Preparation of Various Human Cancer Cell Lines

Human ovarian cancer cell lines (SKOV3, OVCAR3), human breast cancer cell lines (MCF-7, T47D, BT-474), human hepatocarcinoma cell lines (Hep3B, HepG2), human glioblastoma cell lines (U87MG, U251), human colorectal cancer cell lines (SW480, HT-29, HCT116, Caco-2), human lung cancer cell lines (A549, NCIH358, NCI-H460) and a human prostate cancer cell line (22RV1), a human cervical cancer cell line (HeLa), a human melanoma cell line (A375), and a human stomach cancer cell line (NCI-N87) were purchased from Korean Cell Line Bank (KCLB). It was confirmed that all cancer cells had no mycoplasma using e-Myco mycoplasma PCR detection kit (iNtRON Biotechnology).

2-2: Method for Forming Spheroids

Cancer cells (1×10⁶) were inoculated on carious polymer thin film substrates, and cultured appropriately in RPMI-1640 medium, DMEM (Dulbecco's Modified Eagle Medium) medium, or MEM (Minimal Essential Medium) medium, comprising 10% (v/v) serum replacement (SR, Gibco), 1% (v/v) penicillin/streptomycin (P/S, Gibco) and L-glutamine, under the humidified 5% CO2 atmosphere of 37° C.

Specifically, SKOV3, T47D, BT-474, SW480, HT29,22RV1, A549, NCI-H358, NCI-N87, OVCAR3, NCI-H460, and HCT116 cell lines were cultured in RPMI-1640 medium (Gibco) comprising 10% (v/v) SR, 1% (v/v) P/S, and 25 mM HEPES (Gibco). MCF-7, Hep3B, HeLa, U251, and A375 cell lines were cultured in DMEM comprising 10% (v/v) SR and 1% (v/v) P/S(Gibco). HepG2, U87MG, and Caco-2 cell lines were cultured in MEM comprising 10% (v/v) SR and 1% (v/v) P/S (Gibco). In addition, for optimal growth of spheroids, the medium was replaced ever 2-3 days.

2-3: Confirmation of Specificity of Spheroid Formation of Cyclosiloxane Polymer Thin Films

To introduce various surface functionality on a cell culture substrate, a library of polymer thin films (PTFs) was constructed on conventional tissue culture plates (TCP) from various monomers using iCVD (initiated chemical vapor deposition) process, and the manufacturing capacity of cancer-forming spheroids of each PTF was confirmed (FIG. 1m). For this, the human cervical cancer cell line, SKOV3 was cultured in various PTFs. The chemical structures composing tested PTFs were shown in FIG. 1a to FIG. 1f. FIG. 1a shows the structure of EGDMA (ethylene glycol diacrylate) and its polymer (pEGDMA), and FIG. 1b shows the structure of VIDZ (1-vinyl imidazole) and its polymer (pVIDZ), and FIG. 1c shows the structure of IBA (isobornyl acrylate) and its polymer (pIBA), and FIG. 1d shows the structure of PFDA (1H,1H,2H,2H-perfluorodecyl acrylate) and its polymer (pPFDA), and FIG. 1e shows the structure of GMA (glycidyl methacrylate) and its polymer (pGMA), and FIG. 1f shows the structure of V4D4 (2,4,6,8-tetravinyl-2,4,6,8-tetramethyl cyclotetrasiloxane) and its polymer (pV4D4).

As a result, it was confirmed that a very large number of multicellular spheroids were formed within 24 hours only on pV4D4 [poly(2,4,6,8-tetravinyl-2,4,6,8-tetramethyl cyclotetrasiloxane)] PTF prepared by a cyclosiloxane compound polymer. In contrast thereto, SKOV3 grown on other PTFs showed a form of spreading by being attached similarly to cells grown on TCP (FIG. 1n). FIG. 1n is a drawing which confirms formation of cancer-forming spheroids on conventional TCP and various functional PTFs.

### Example 3: Confirmation of Possibility of Spheroid Formation of Substrate Comprising Various Cyclosiloxane Compounds

To confirm whether spheroids are formed on a cell culture substrate comprising various cyclosiloxane compounds, SKOV3 cells were inoculated on the cell culture substrate produced in Example 1-2 and in 24 hours, whether spheroids were formed was confirmed.

Specifically, as the result of confirming whether spheroids were formed on the cell culture substrate comprising various cyclosiloxane compounds of FIG. 1g to FIG. 1l, it was confirmed that spheroids were formed even on the cell substrate comprising 1,3,5-trivinyl-1,3,5-trimethylcyclotrisiloxane (FIG. 1g), 2,4,6,8-tetramethyl-2,4,6,8-tetravinylcyclotetrasiloxane (V4D4) (FIG. 1h), 2,4,6,8,10-pentamethyl-2,4,6,8,10-pentavinylcyclopentasoxane (FIG. 1i), 2,4,6,8,10,12-hexamethyl-2,4,6,8,10,12-hexavinyl-cyclohexasiloxane (FIG. 1j), octa(vinylsilasesquioxane) (FIG. 1k), and 2,2,4,4,6,6,8,8,10,10,12,12-dodecamethylcyclohexasiloxane (FIG. 1l) (FIG. 1o to FIG. 1t).

FIG. 1o to FIG. 1t show spheroids formed on the substrate comprising various cyclosiloxane compounds, and FIG. 1o shows spheroids formed on the cell culture substrate comprising 1,3,5-trivinyl-1,3,5-trimethylcyclotrisiloxane, and FIG. 1p shows spheroids formed on the cell culture substrate comprising 2,4,6,8-tetramethyl-2,4,6,8-tetravinylcyclotetrasiloxane (V4D4), and FIG. 1q shows spheroids formed on the cell culture substrate comprising 2,4,6,8,10-pentamethyl-2,4,6,8,10-pentavinylcyclopentasoxane, and FIG. 1r shows spheroids formed on the cell culture substrate comprising 2,4,6,8,10,12-hexamethyl-2,4,6,8,10,12-hexavinyl-cyclohexasiloxane, and FIG. is shows spheroids formed on the cell culture substrate comprising octa(vinylsilasesquioxane), and FIG. 1t shows spheroids formed on the cell culture substrate comprising 2,2,4,4,6,6,8,8,10,10,12,12-dodecamethyl cyclohexasiloxane.

### Example 4: Formation of Possibility of Spheroid Formation Using Various Cancer Cell Lines

Whether PTFs comprising a cyclosiloxane compound polymer had spheroid-forming enhancing ability even in other cancer cell lines other than the human ovarian cancer cell line SKOV3 was confirmed.

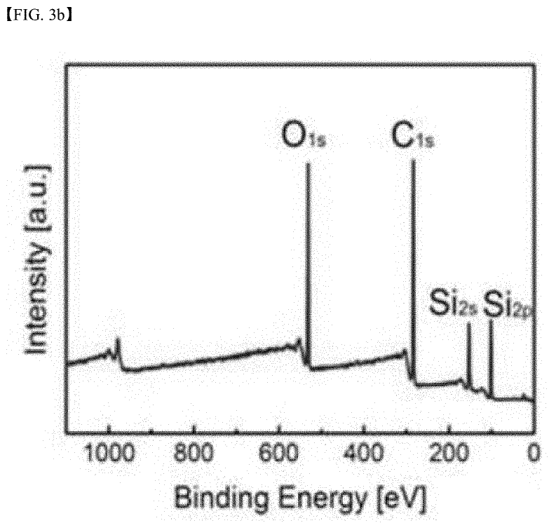

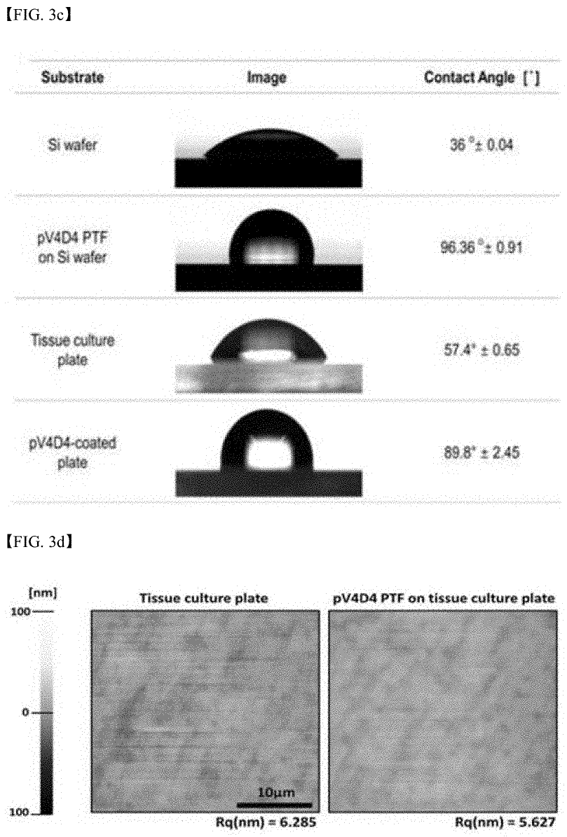

As a result, multicellular spheroids (˜50-300 μm diameter) were formed in most of human cancer cell lines within 24 hours regardless of roots or origins, and showed high efficiency and reproducibility (FIG. 2a). The shape of each spheroid varied from the shape of a ‘grape cluster’ to a dense sphere (FIG. 2b), and this result indicates the diversity of the PTF platform.

### Comparative Example 1: Conventional Method for Forming Spheroids

To form spheroids by conventional methods, it was performed as follows.

Specifically, Hanging-drop 96-well plate (3D Biomatrix), U-bottom 96-well plate (SBio), and ultra-low-attachment (ULA) 6-well plate (Corning) were used. Cells were inoculated on hanging drop plate at a density of 1×10⁴ cells/50 μl, and inoculated on U-bottom plate at a density of 5×10⁴ cells/2 ml, and inoculated on ULA plate at a density of 5×10⁵ cells/2 ml. For optimal growth of spheroids, the medium was replaced every 2-3 days.

### Example 5: Analysis of Characteristics of Prepared Cancer Stem Cell Spheroids

5-1: Characteristic of Forming Cancer Cell-Derived Spheroids of Cyclosiloxane Compound Polymer Substrate

In the process of spheroid formation of Example 2-3, each cancer cell was attached on pV4D4 surface at first, but immediately multicellular spheroids were formed simultaneously by intercellular interaction. The activated intercellular interaction on the pV4D4 is a phenomenon which is not observed in other spheroid-forming technology, dependent on simple physical or mechanical contact-based binding.

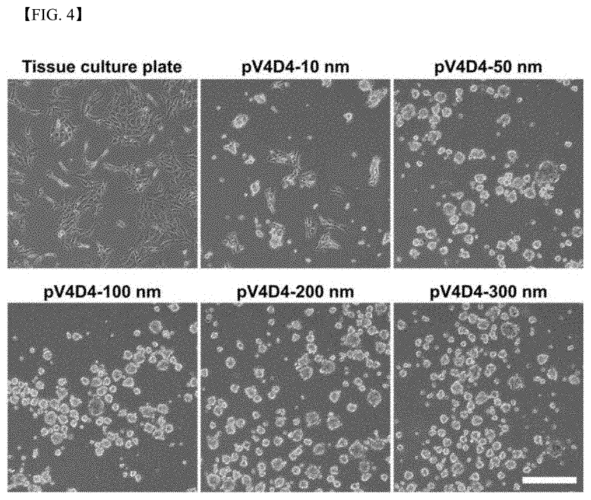

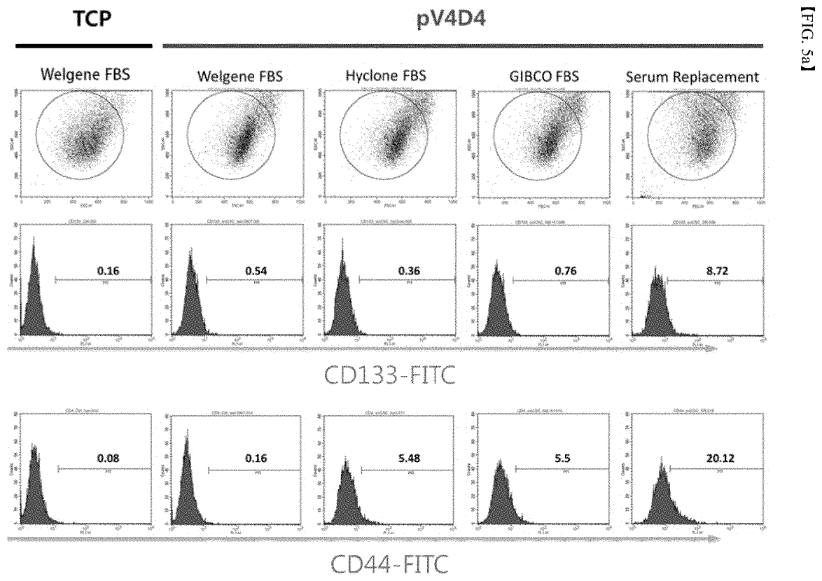

Different from the conventional hydrophilic ULA (ultra-low-attachment) surface, the pV4D4 PTF surface (FIGS. 3a and b, Table 2), characterized by FT-IR (Fourier transform infrared) spectroscopy and XPS (X-ray photoelectron spectroscopy), is relatively hydrophobic with the water contact angle of 90° (FIG. 3c), and has a smooth surface with roughness similar to conventional TCPs (FIG. 3d).

TABLE 2

| Atoms | Measured value [%] | Theoretical value [%] |
|---|---|---|
| C | 59.08 | 60 |
| O | 21.49 | 20 |
| Si | 19.42 | 20 |
| Total | 100 | 100 |

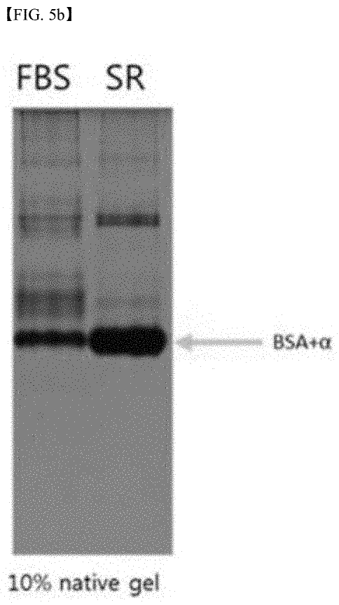

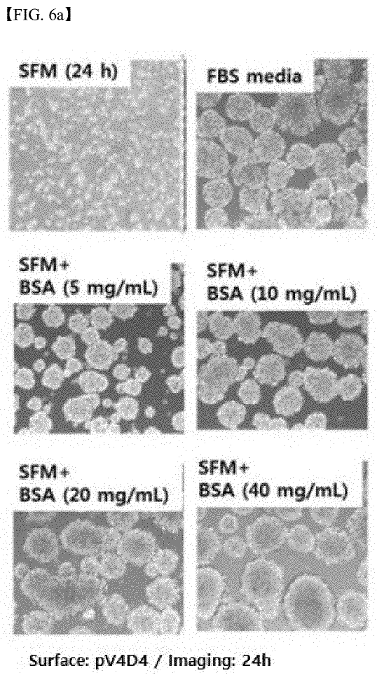

In addition, pV4D4 was deposited on TCP with a thickness of 10, 50, 100, 200 and 300 nm using an He—Ne laser (JDS Uniphase) interferometer system to produce pV4D4 PTFs with various thickness, and the correlation of the thickness and spheroid formation ability was confirmed, and the change of thickness of pV4D4 PTFs in the range of 50 to 300 nm did not affect the spheroid formation ability at all (FIG. 4). Taking the results together, it can be seen that in case of pV4D4, the specific surface functionality (chemical or biological stimulus) present in pV4D4, not a mechanical signal, induces spheroid formation.

These results suggest that cell culture substrates comprising a polymer formed by cyclosiloxane compound can form 3D spheroids having a specific property from cancer cells.

5-2: Analysis of Shapes of Prepared Cancer Stem Cell Spheroids

At first, characteristics of cancer cell spheroids prepared by culturing in the pV4D4 PTF for 4 to 8 days were compared with spheroids prepared by conventional other spheroid-forming method prepared in 1-2.

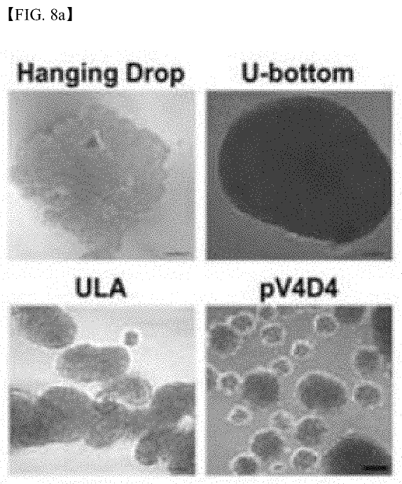

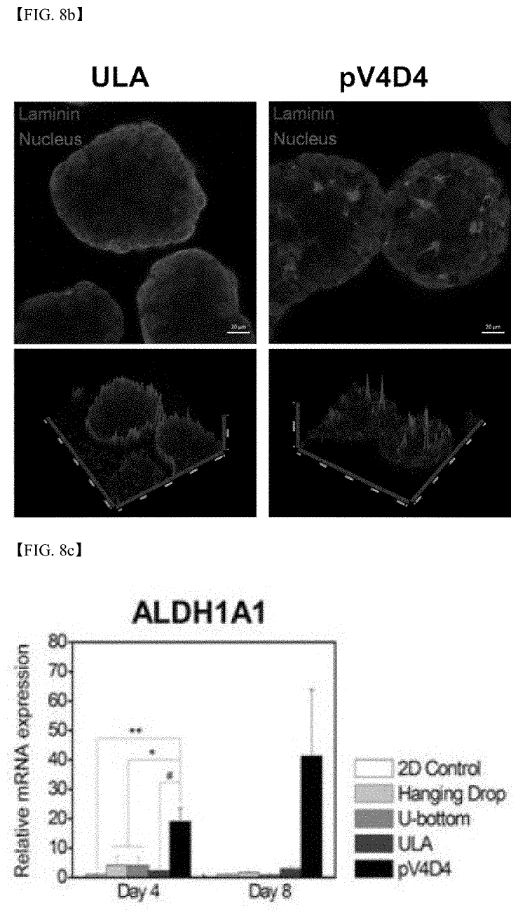

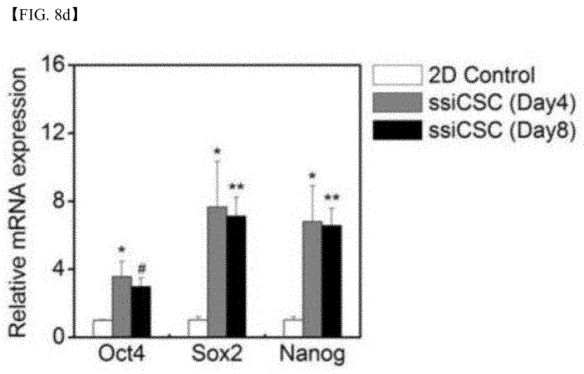

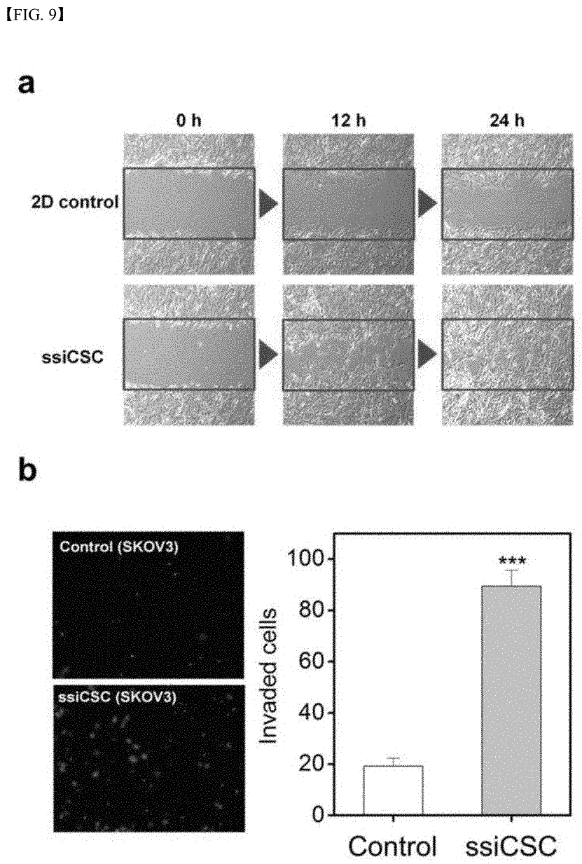

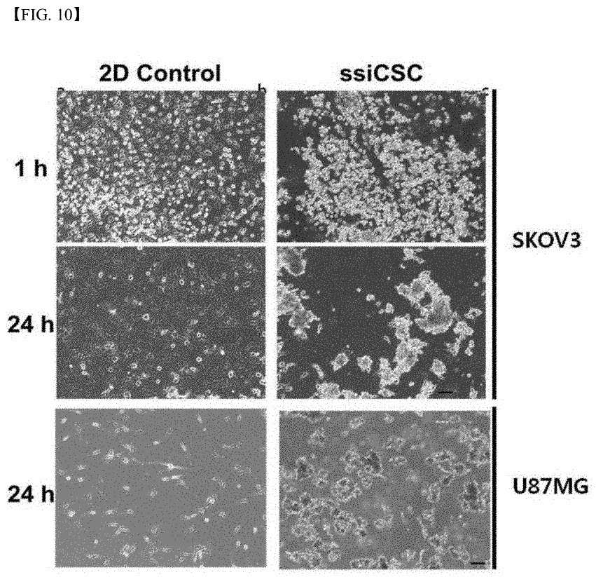

As a result, SKOV3 cancer cell formed one big aggregated spheroid by the hanging-drop method and U-bottom method, but formed several small spheroids on the ULA and pV4D4 surface, and the spheroids formed on the pV4D4 were more homogeneous and slightly smaller than the spheroids formed on the ULA (FIG. 8a). In addition, as the result of comparing SKOV3 spheroids cultured on the ULA surface or pV4D4 surface for 8 days by immunocytochemistry analysis, in case of spheroids cultured on the pV4D4 surface, laminin which is a major component of extracellular matrix (ECM) was present inside of the spheroids, but in case of spheroids cultured on the ULA surface, laminin was present only around the spheroids (FIG. 8b).

Based on the result, it is shown that the spheroids prepared by culturing in pV4D4 of the present invention are not cancer cell aggregates such as spheroids prepared using the conventional method, and repeat the ECM-mediated multicellular structure of tumor tissue in vivo. It is shown that the ECM plays a critical role in the development of drug resistance, self-regeneration and cancer-formation ability in the tumor microenvironment.

### Example 6: Preparation of Cancer Stem Cell Spheroids Using Albumin

6-1: Preparation of Cancer Stem Cell Spheroids

To form cancer stem cell spheroids, SKOV3 cells (1×10⁶) were inoculated on a substrate coated by pV4D4, and suitably cultured on RPMI-1640 comprising 10% (v/v) serum replacement (SR, Gibco), 1% (v/v) penicillin/streptomycin (P/S, Gibco) and L-glutamine under the humidified 5% CO2 atmosphere of 37° C. For optimal growth of spheroids, the medium was replaced every 2-3 days, and spheroids were obtained. The albumin concentration of the serum replacement was 1 mg/ml or more, and was higher than the concentration of the albumin comprised in FBS (fetal bovine serum) serum.

6-2: Confirmation of Cancer Stem Cell Spheroid Formation Through Confirmation of CSC-Related Gene Expression

To confirm whether spheroids prepared in Example 6-1 have properties of cancer stem cells, expression of CSC-related genes was confirmed using qRT-PCR and RT-PCR. As a control group, spheroids formed by the conventional method of Comparative example 1 was used.

Specifically, to perform qRT-PCR, according to the manufacturer's instructions, total RNA was isolated from 2D-cultured control cancer cells and ssiCSC spheroids. The isolated total RNA was mixed with AccuPower RT PreMix (Bioneer) and was under reverse transcription to cDNA using Rotor-Gene Q thermocycler (Qiagen). The qRT-PCR experiment was performed with 50 ng RNA using Rotor-Gene Q thermocycler (Qiagen) and KAPA SYBR FAST Universal qPCR kit (Kapa Biosystems) according to the manufacturer's instructions.

In addition, to analyze the expression level of CD44, CD133, ALDH1A1, ALDH1A2 and EpCAM that are cancer stem cell marker genes using RT-PCR, 30 cycle program was performed using HyperScript One-step RT-PCR kit (GeneAll Biotechnology Co. Ltd.) according to the manufacturer's instructions. β-actin was used as an internal control.

The sequences of primers for performing qRT-PCR and RT-PCR were shown in the following Table 1.

| Gene | | | |
|---|---|---|---|
| (Accession number) | Primer pair | Primer sequence | SEQ ID NO. |
| Human β-actin | Forward primer | GTCTTCCCCTCCATCGTG | 1 |
| (NM_001101.3) | Reverse primer | AGGTGTGGTGCCAGATTTTC | 2 |
| Human ALDH1A1 | Forward primer | CGCCAGACTTACCTGTCCTA | 3 |
| (NM_000689.4) | Reverse primer | GTCAACATCCTCCTTATCTCCT | 4 |
| Human ALDH1A2 | Forward primer | CAGCTTTGTGCTGTGGCAAT | 5 |
| (NM_003888.3) | Reverse primer | GGAAAGCCAGCCTCCTTGAT | 6 |
| Human EpCAM | Forward primer | AGTTGGTGCACAAAATACTGTCAT | 7 |
| (NM_002354.2) | Reverse primer | TCCCAAGTTTTGAGCCATTC | 8 |
| Human CD44 | Forward primer | TCCAACACCTCCCAGTATGA | 9 |
| (NM_006718390.3) | Reverse primer | GGCAGGTCTGTGACTGATGT | 10 |
| Human CD90 | Forward primer | AGAGACTTGGATGAGGAG | 11 |
| (NM_001311162.1) | Reverse primer | CTGAGAATGCTGGAGATG | 12 |
| Human CD133 | Forward primer | ACCAGGTAAGAACCCGGATCAA | 13 |
| (NM_006713974.3) | Reverse primer | CAAGAATTCCGCCTCCTAGCACT | 14 |
| Human LGR5 | Forward primer | CCTGCTTGACTTTGAGGAAGACC | 15 |
| (NM_001277227.1) | Reverse primer | CCAGCCATCAAGCAGGTGTTCA | 16 |
| Human Oct3/4 | Forward primer | CTTGCTGCAGAAGTGGGTGGAGGAA | 17 |
| (NM_001285987.1) | Reverse primer | CTGCAGTGTGGGTTTCGGGCA | 18 |
| Human Sox2 | Forward primer | CATCACCCACAGCAAATGACA | 19 |
| (NM_003106.3) | Reverse primer | GCTCCTACCGTACCACTAGAACTT | 20 |
| Human Nanog | Forward primer | AATACCTCAGCCTCCAGCAGATG | 21 |
| (NM_011520852.1) | Reverse primer | TGCGTCACACCATTGCTATTCTTC | 22 |
| Human ABCB1 | Forward primer | TGACATTTATTCAAAGTTAAAAGCA | 23 |
| (NM_001348946.1) | Reverse primer | TAGACACTTTATGCAAACATTTCAA | 24 |
| Human ABCB2 | Forward primer | CGTTGTCAGTTATGCAGCGG | 25 |
| (NM_000593.5) | Reverse primer | ATAGATCCCGTCACCCACGA | 26 |
| Human ABCB5 | Forward primer | CACAAAAGGCCATTCAGGCT | 27 |
| (NM_011515367.2) | Reverse primer | GCTGAGGAATCCACCCAATCT | 28 |
| Human ABCC1 | Forward primer | GGAATACCAGCAACCCCGACTT | 29 |
| (NM_017023243.1) | Reverse primer | TTTTGGTTTTGTTGAGAGGTGTC | 30 |
| Human ABCG2 | Forward primer | TCATGTTAGGATTGAAGCCAAAGGC | 31 |
| (NM_001348989.1) | Reverse primer | TGTGAGATTGACCAACAGACCTGA | 32 |
| Human DKK1 | Forward primer | TCCCCTGTGATTGCAGTAAA | 33 |
| (NM_012242.2) | Reverse primer | TCCAAGAGATCCTTGCGTTC | 34 |
| Human β-catenin | Forward primer | ACAGCTCGTTGTACCGCTGG | 35 |
| (NM_001330729.1) | Reverse primer | AGCTTGGGGTCCACCACTAG | 36 |
| Human AXIN2 | Forward primer | AGTGTGAGGTCCACGGAAAC | 37 |
| (NM_017025194.1) | Reverse primer | CTTCACACTGCGATGCATTT | 38 |
| Human MMP-2 | Forward primer | TCTCCTGACATTGACCTTGGC | 39 |
| (NM_001302510.1) | Reverse primer | CAAGGTGCTGGCTGAGTAGATC | 40 |

As a result, it was confirmed that the expression of ALDH1A1 (aldehyde dehydrogenase 1 family member A1) known as a CSC marker was largely increased only in SKOV3 spheroids prepared by culturing in pV4D4, among various spheroid forming methods through quantitative real-time PCR (quantitative real-time PCR polymerase chain reaction; qRT-PCR) analysis (FIG. 8c). In addition, it was confirmed that in SKOV3 spheroids prepared by culturing in pV4D4, the expression of Oct3/4, Sox2 and Nanog that are typical self-regenerative genes was significantly increased, compared to the 2D-cultured SKOV3 control group grown on TCP (FIG. 8d). Through the result, it could be seen that the cancer cells in the spheroids had stem cell characteristics.

6-3: Confirmation of Cancer Stem Cell Inducing Function of Albumin

To confirm that the cancer stem cell (CSC) characteristics of spheroids were induced by albumin, the following experiment was performed.

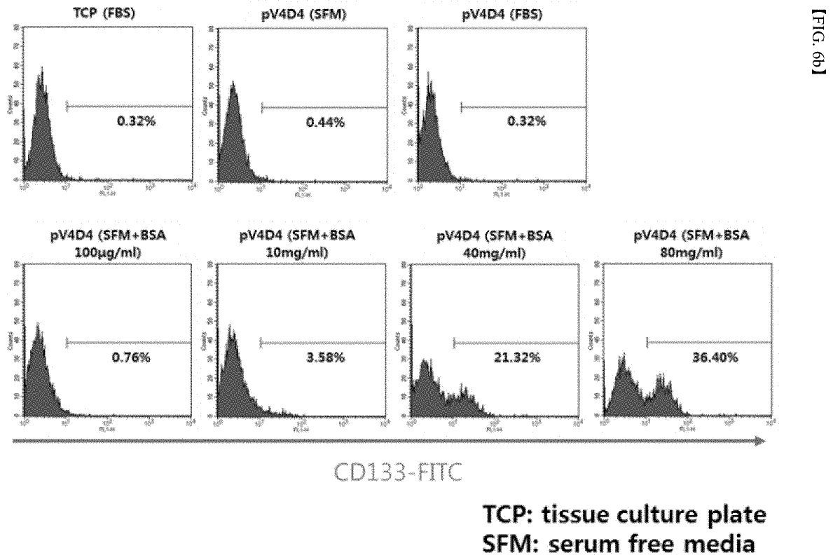

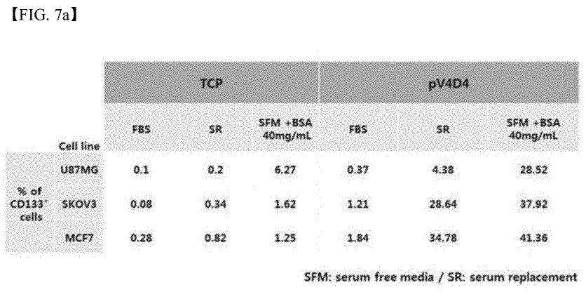

At first, when various kinds of FBSs and serum replacements (SR) were used, to confirm the expression level of CSC marker genes, the following experiment was performed. Specifically, after culturing U87MG plated on the pV4D4 PTF in 3 kinds (Welgene, Hyclone, GIBCO) of FBSs and SRs for 6 days, the expression level of the CSC markers, CD133 and CD44 was confirmed by flow cytometry. As a result, it was confirmed that the expression level of CD133 and CD44 in case that SR was added was excellent than 3 kinds of FBS (FIG. 5a). In addition, as the result of comparing the albumin content of FBS and SR using native-gel, it was confirmed that SR comprised more amount of albumin than FBS (FIG. 5b). Based on the result, it can be seen that SR promotes CSC induction of spheroids, as it has higher albumin content than FBS. Then, after culturing U85MG of 5×10⁵ plated on the pV4D4 PTF in a serum-free medium (SFM) comprising FBS and various concentrations of bovine serum albumin (BSA) (0.1, 5, 10, 20, 40, and 80 mg/ml) for 8 days, the spheroid formation was confirmed, and the expression level of the CSC marker gene (CD133) of the cell cultured in the serum-free medium (SFM) at a BSA concentration of 0.1, 5, 10, 20, 40, and 80 mg/ml was confirmed.

As a result, it was confirmed that spheroids were formed in a BSA-comprising medium, and it was confirmed that the CSC marker, CD133 was expressed (FIGS. 6a and b). In addition, it was confirmed that the expression level of CD133 was increased, as the concentration of BSA was raised. Furthermore, it was confirmed that spheroids were formed but CD133 which is the CSC marker was not expressed, when FBS which is comprised in a general cell growth medium was used. In other words, it could be seen that characteristics of cancer stem cells were shown as the CSC marker was expressed under the medium comprising albumin at a specific concentration or higher, but the CSC marker was not expressed in case that the albumin was comprised at a low concentration, and therefore they did not have characteristic of cancer stem cells, and thereby it was confirmed that cancer stem cells were induced by albumin at a specific concentration or higher.

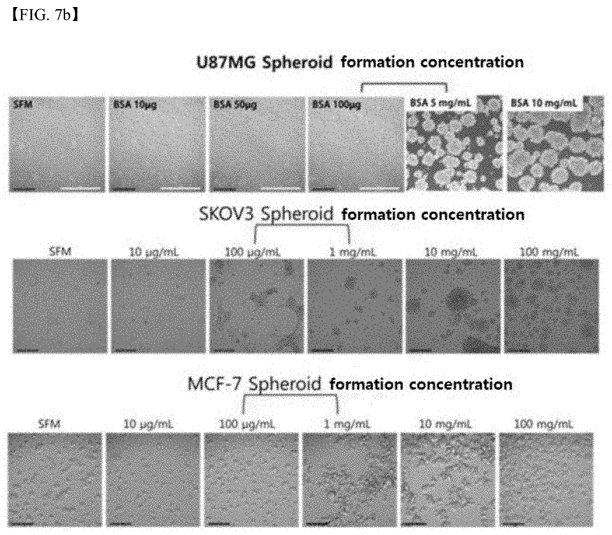

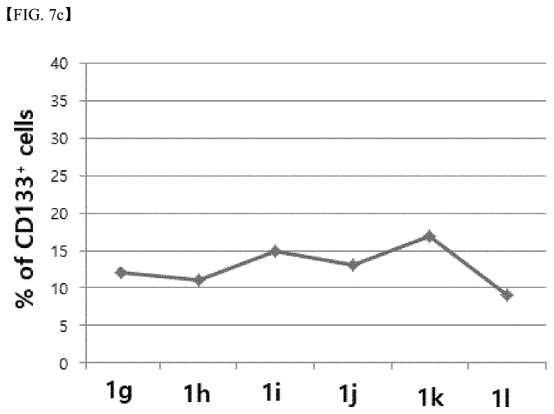

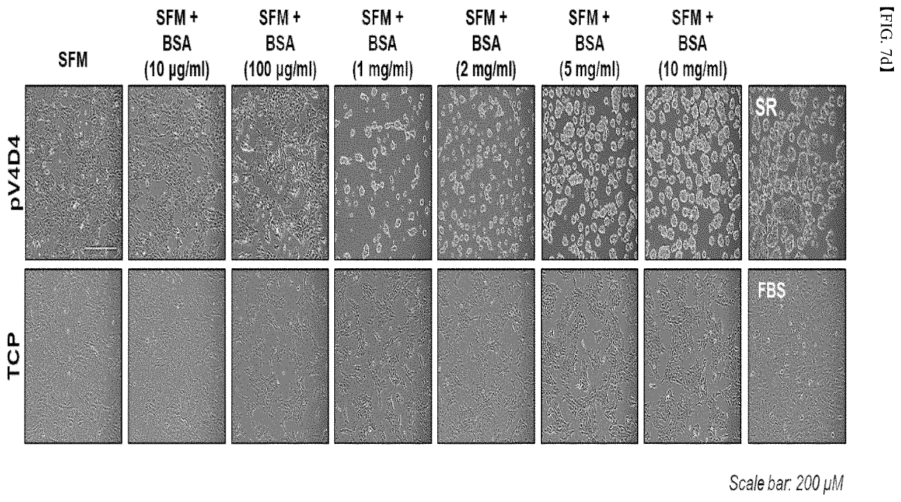

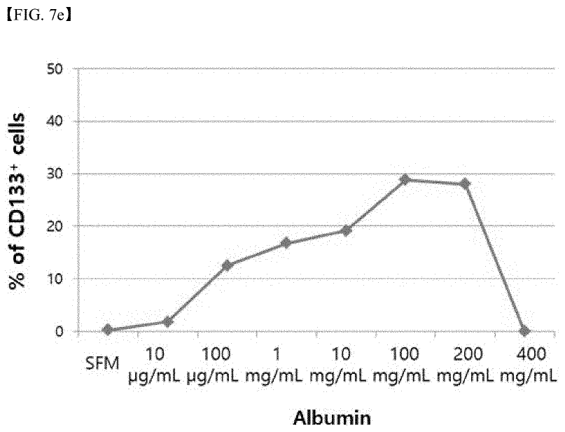

In addition, when U87MG, SKOV3, and MCF7 were cultured in a serum-free medium (SFM) comprising FBS, SR or 40 mg/ml BSA in TCP and pV4D4 PTF, the expression level of the CSC marker, CD133 was confirmed by flow cytometry, and represented by a chart (FIG. 7a and FIG. 7b).

Based on the result, it could be seen that albumin could induce cancer stem cells, and when cultured on the pV4D4 PTF, culturing by comprising albumin at a specific concentration or higher in a serum-free medium (SFM) could induce cancer stem cells efficiently.

6-4: Confirmation of Cancer Stem Cell Characteristics of Spheroids Prepared in Substrates Comprising Various Cyclosiloxane Compounds

To confirm whether spheroids prepared in substrates comprising various cyclosiloxane compounds have cancer stem cell characteristics, the expression level of the cancer stem cell marker gene, CD133 was measured, and the result was shown in FIG. 7c.

Specifically, using pV4D4 and 6 kinds of cyclosiloxane compounds of FIG. 1g to FIG. 1l, copolymer substrates were formed at a ratio of 9:1, respectively. FIG. 1g shows 1,3,5-trivinyl-1,3,5-trimethylcyclotrisiloxane, and FIG. 1h shows 2,4,6,8-tetramethyl-2,4,6,8-tetravinylcyclotetrasiloxane (V4D4), and FIG. 1i shows 2,4,6,8,10-pentamethyl-2,4,6,8,10-pentavinylcyclopentasiloxane, and FIG. 1j shows 2,4,6,8,10,12-hexamethyl-2,4,6,8,10,12-hexavinyl-cyclohexasiloxane, and FIG. 1k shows octa(vinylsilasesquioxane), and FIG. 1l shows 2,2,4,4,6,6,8,8,10,10,12,12-dodecamethylcyclohexasiloxane. SKOV3 cells were treated to each substrate, and in 24 hours, it was confirmed that spheroids were formed, and in 8 days, it was confirmed that the number of cells expressing CD133 was increased by flow cytometry.

In the axis of FIG. 7c, 1g shows the CD133 expression of cancer stem cell spheroids prepared in the substrate in which pV4D4 and the cyclosiloxane compound of FIG. 1g were copolymerized, and 1h shows the CD133 expression of cancer cell spheroids prepared in the substrate in which pV4D4 and the cyclosiloxane compound of FIG. 1h were copolymerized, and 1i shows the CD133 expression of cancer stem cell spheroids prepared in the substrate of pV4D4 and the cyclosiloxane compound of FIG. 1i were copolymerized, and 1j shows the CD133 expression of cancer stem cell spheroids prepared in the substrate of pV4D4 and the cyclosiloxane compound of FIG. 1j were copolymerized, and 1k shows the CD133 expression of cancer stem cell spheroids prepared in the substrate of pV4D4 and the cyclosiloxane compound of FIG. 1k were copolymerized, and 11 shows the CD133 expression of cancer stem cell spheroids prepared in the substrate of pV4D4 and the cyclosiloxane compound of FIG. 1l were copolymerized.

Thus, it could be confirmed that cancer stem cell characteristics could be induced even when other cyclosiloxane compounds other than pV4D4 were used.

### Example 7: Cancer Stem Cell Spheroids at Various Albumin Concentrations

7-1: Confirmation of Spheroid Formation at Various Albumin Concentrations

The medium was composed by adding BSA so that the concentration of albumin was 0, 0.01 mg/ml, 0.1 mg/ml, 1 mg/ml, 2 mg/ml, 5 mg/ml, and 10 mg/ml to an SFM medium, and by culturing cancer cells in a substrate comprising a cyclosiloxane compound and a TCP substrate, whether spheroids were formed was confirmed.

As a result, as could be seen in FIG. 7d, the spheroid shape was shown in the cyclosiloxane compound, pV4D4 substrate, but spheroids were not formed in the TCP substrate.

7-2: Confirmation of Cancer Stem Cell Markers of Spheroids

The medium was composed by adding BSA so that the concentration of albumin was 0, 0.01 mg/ml, 0.1 mg/ml, 1 mg/ml, 10 mg/ml, 100 mg/ml, 200 mg/ml, 400 mg/ml to an SFM medium, and by culturing cancer cells in a substrate comprising a cyclosiloxane compound, whether spheroids were formed was confirmed.

As a result, as could be seen in FIG. 7e, it could be confirmed that the expression level of CD133 was changed according to the albumin concentration.

Taking the results together, it can be seen that one example of polymers formed by cyclosiloxane compounds, the pV4D4 surface provides a specific stimulus which activates and modifies SKOV3 cancer cells to induce formation of spheroids of cancer cells, and albumin induces their cancer stem cell characteristics, thereby forming spheroids comprising a significantly large amount of CSC-like cells. Accordingly, the CSC-like cells were named surface-stimuli-induced cancer stem cells (ssiCSCs).

### Example 8: Confirmation of Cancer Stem Cell Spheroid Formation Ability Using Various Cancer Cell Lines

To confirm the possibility of generalization of the method for preparing spheroids using the pV4D4, ssiCSC spheroids derived from various cancer cell lines were prepared, and the CSC-related characteristics were confirmed. For this, 4 kinds of human cancer cell lines derived from various tissues were selected: SKOV3, MCF-7 (human breast cancer), Hep3B (human liver cancer) and SW480 (human colorectal cancer). In addition, estimated CSC characteristics for each cell line were confirmed using specific surface markers by each cell line: SKOV331-ALDH1A1; MCF-7-CD44 (cluster of differentiation 44); Hep3B36-CD90; and SW48037-LGR5 (leucine-rich repeat-containing G-proteincoupled receptor 5). Furthermore, CD133 was used as a general estimated CSC marker for all cell lines. The expression of CSC marker genes was confirmed by confirming ssiCSC spheroids cultured on the pV4D4 surface for 4 days and 8 days by qRT-PCR, and the expression of the corresponding 2D control group cultured with TCP and the CSC marker genes were compared.

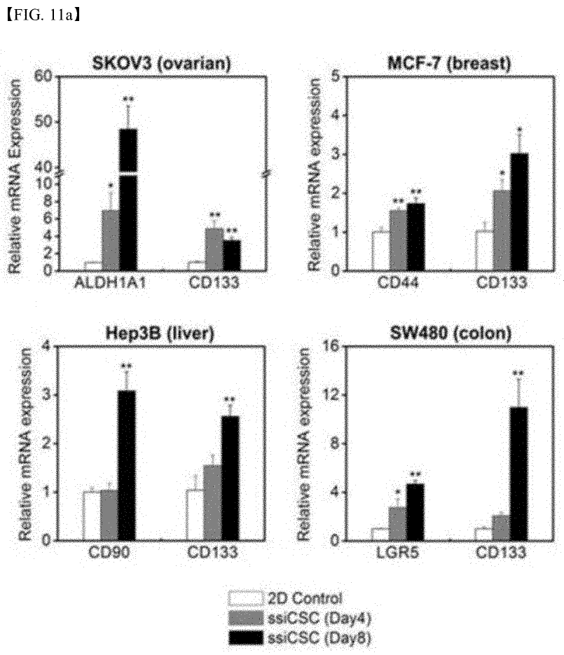

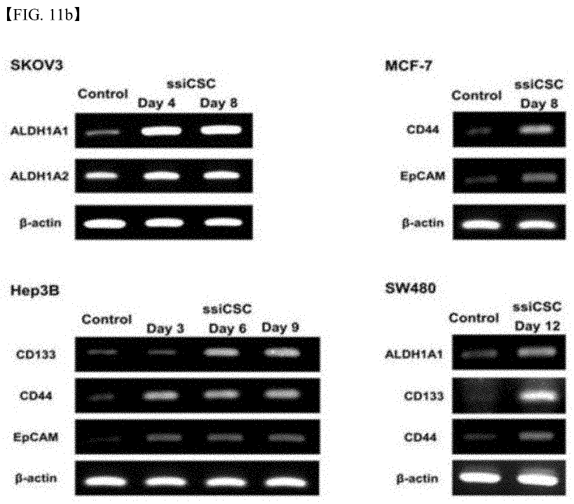

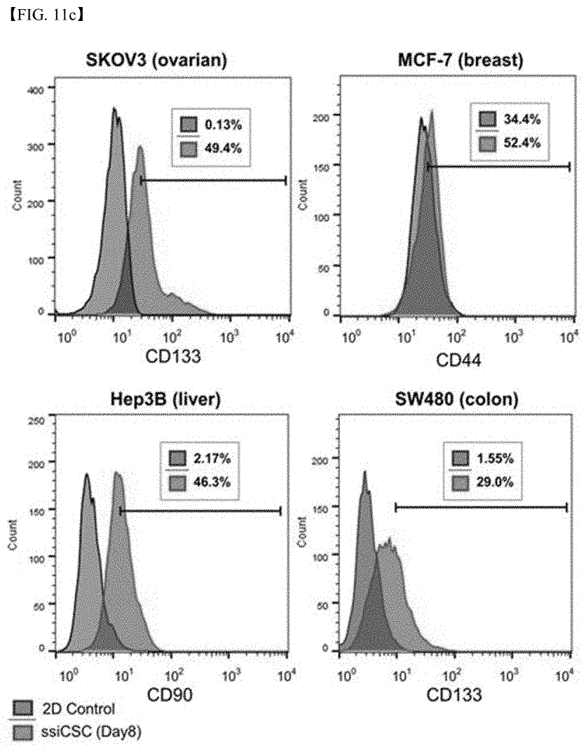

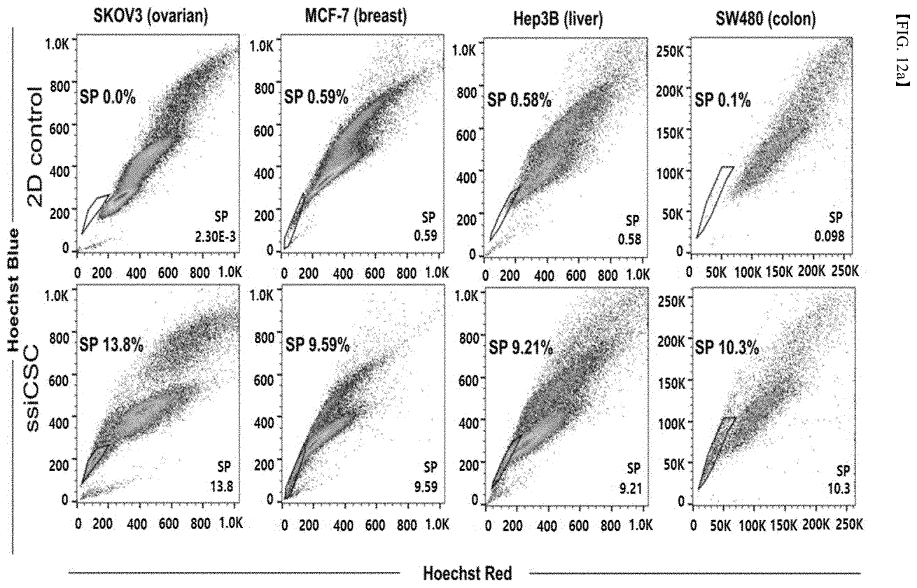

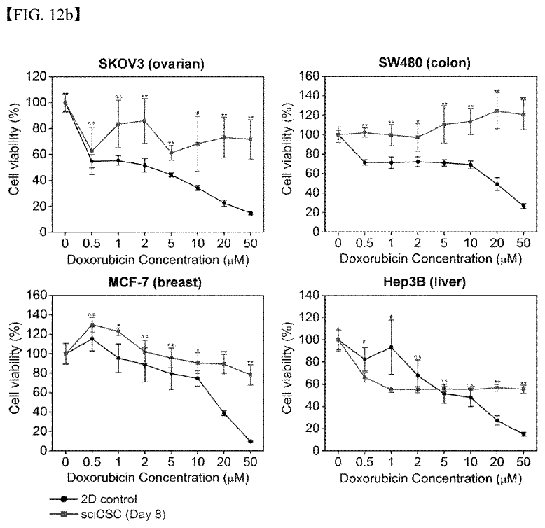

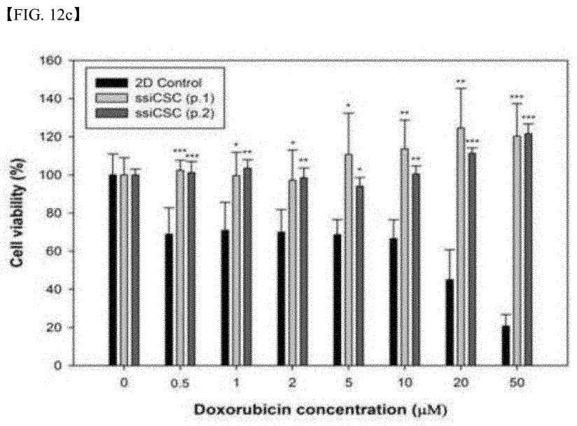

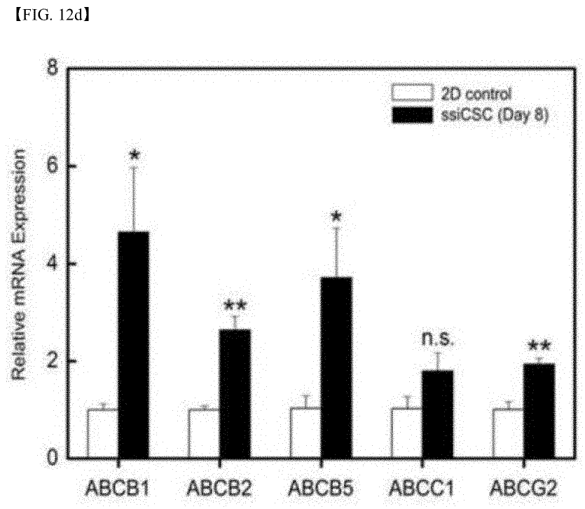

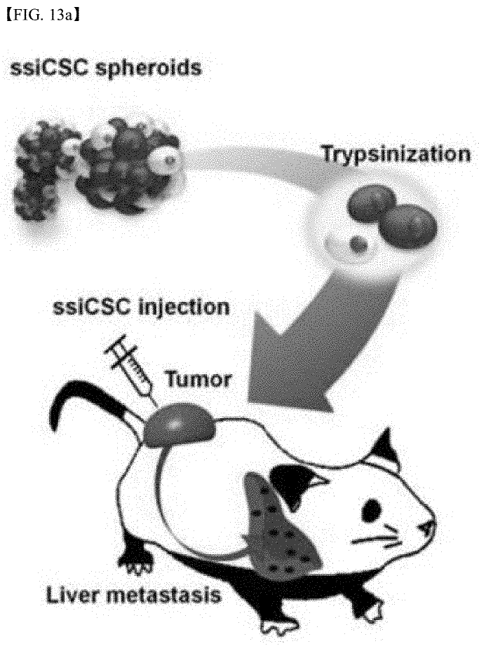

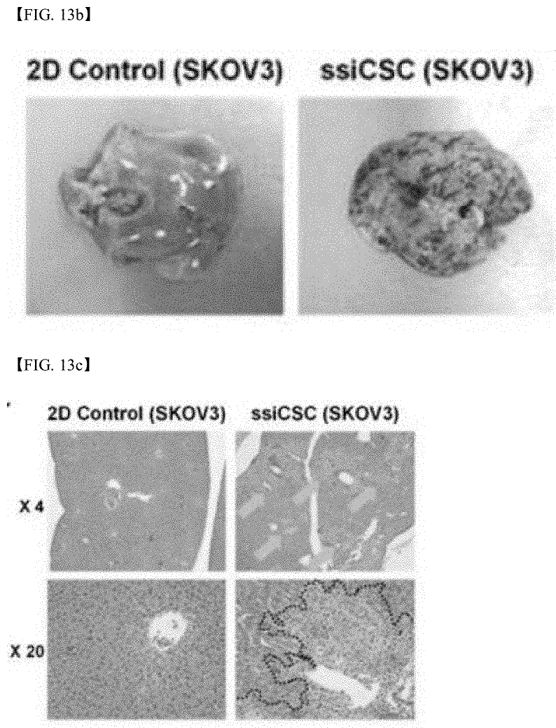

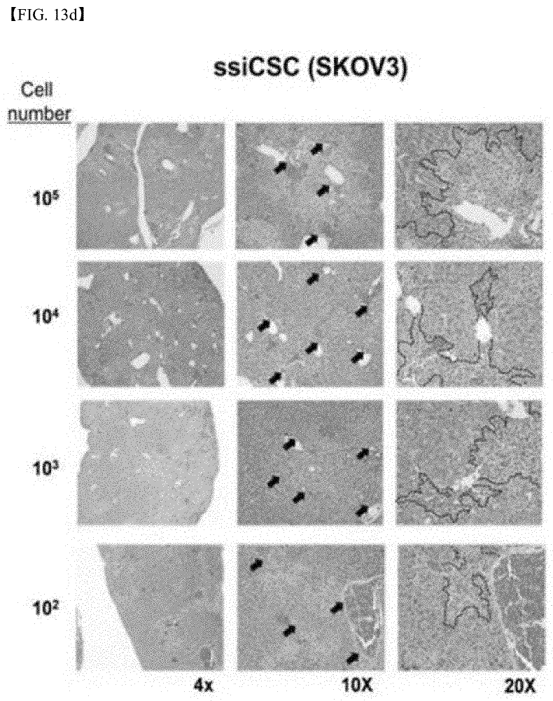

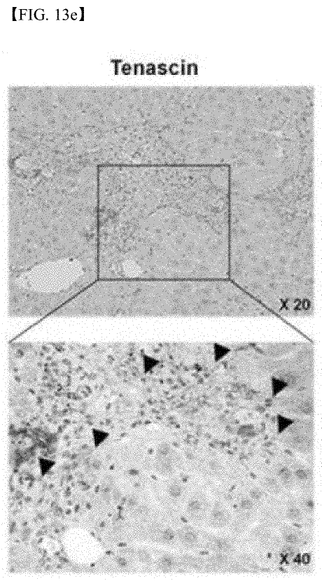

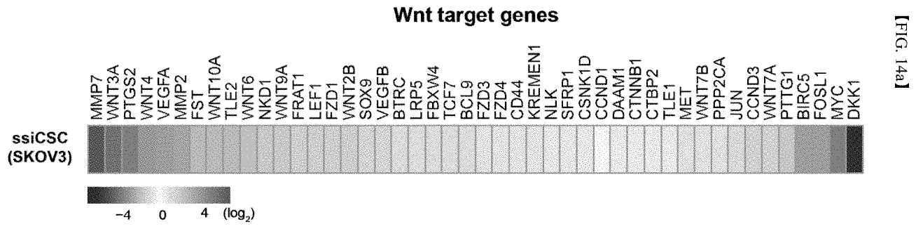

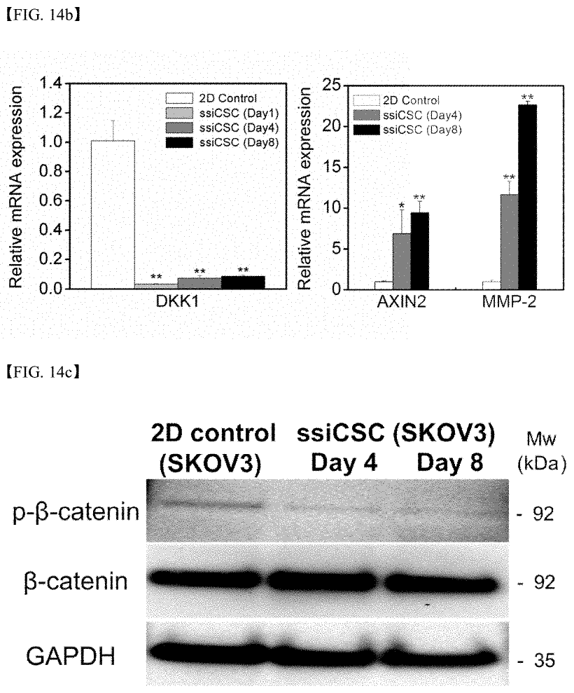

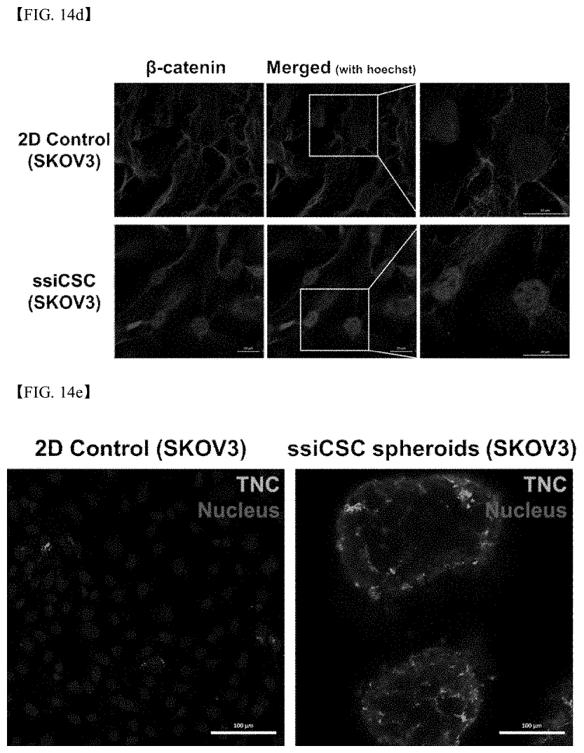

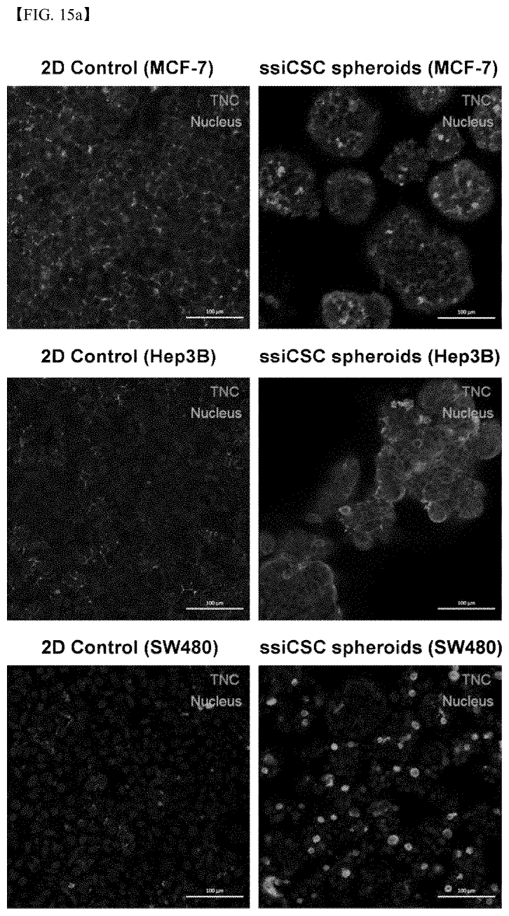

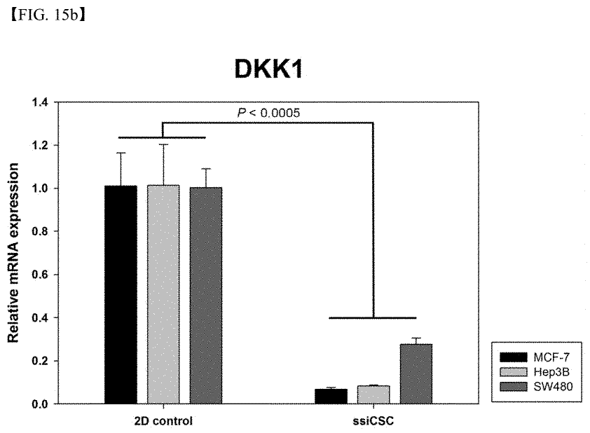

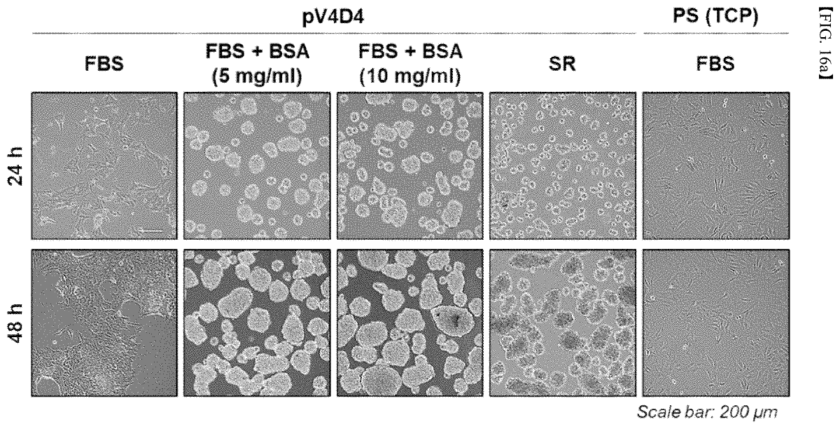

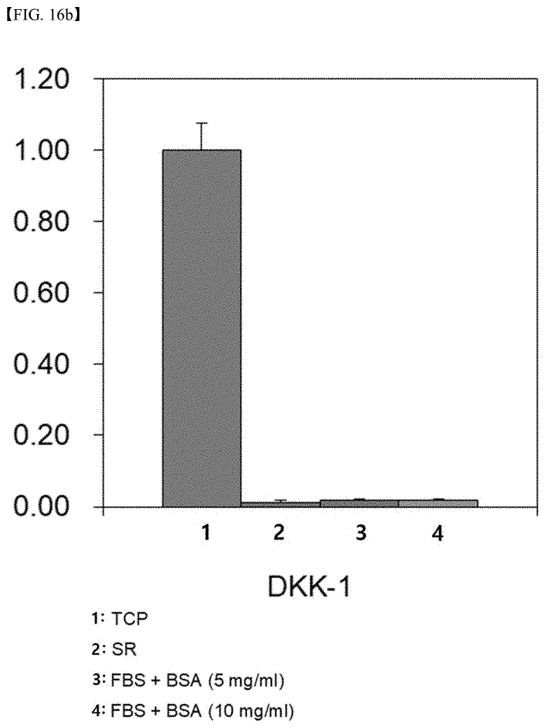

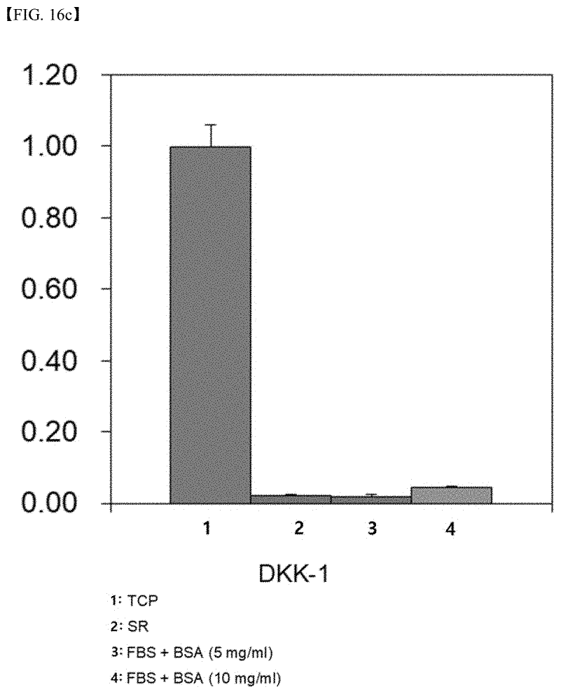

As a result, each cell-type specific CSC marker gene was significantly upregulated in each spheroid, and the expression of the common marker, CD133 was increased in all ssiCSC spheroids (FIG. 11 a). In addition, as the expression level of the marker genes was increased with the culture time, this shows that CSC-like properties are intensified as it is cultured. Furthermore, RTPCR (Reverse transcription-PCR) analysis showed that the expression of various CSC-related genes was increased in all ssiCSC spheroids, compared to the 2D culture control cancer cells (FIG. 11b).

Then, fractions of the CSC-marker-positive cancer cells estimated in spheroids prepared by culturing on the pV4D4 surface for 8 days were quantified by flow cytometry. As a result, it was shown that the expression of cell-type-specific CSC-related surface markers (indicated by gene counts) was increased approximately 10 times in ssiCSC spheroids of SKOV3, Hep3B and SW480, compared to the 2D-cultured control group, and in case of CD44 of MCF-7 cell, it was increased less than 10 times (FIG. 11c).

Such results suggest that ssiCSC spheroids prepared using pV4D4 have properties similar to CSC.

### Example 9: Wound Healing Assay, Invasion Assay and Spheroid Formation Analysis of Prepared Cancer Stem Cell Spheroids

9-1: Analysis Method

SKOV3 cells were cultured in the pV4D4-coated substrate for 8 days. After confirming SKOV3-spheroid formation, the ssiCSC spheroids were isolated with trypsin (TrypLE Express; Gibco) and the isolated cells were washed with D-PBS twice.

Wound healing assay was conducted by densely culturing SKOV3 cells and SKOV3-ssiCSCs in a 6-well plate in a single layer, and then synchronizing the cells in a 1% FBS-containing medium for 24 hours. Then, “wound” was made by uniformly scratching the cell single layer with a standard 200 μl pipette tip. Dropped cells were removed by washing with D-PBS twice, and then a serum-free medium was added. The movement of the cells to the wound region was observed using a phase difference microscope (LumaScope 620, Etaluma) right after the wound was made (0 h), in 12 hours (12 h) and in 24 hours (24 h) after it was made.

Invasion assay was conducted by culturing SKOV3 cells and SKOV3-ssiCSCs cells in a serum-free medium for 24 hours at first, and then culturing in Transwell chamber (Corning). Cells (1×10⁵ cells/well) were plated in the upper chamber of the transparent PET film (8.0 μm pore size) coated with Matrigel (200 μg/ml; Corning), and allowed to penetrate the lower chamber filled with a medium comprising 10% FBS. The cells were cultured for 24 hours and fixed with 4% formaldehyde (Sigma). Cells which did not penetrate on the upper chamber of the film were removed using a cotton swab. Moving cells on the lower surface of the film were stained with Hoechst 33342 (ThermoFisher Scientific), and the nuclei of penetrated cells were counted using a fluorescence microscope (Eclipse 80i, Nikon). Penetration was calculated by the mean cell number per 5 fields of each film.

For spheroid formation assay, SKOV3 cells and SKOV3-ssiCSCs were cultured in DMEM/F12 (1:1, Gibco) comprising B27 (Invitrogen), 20 ng/ml EGF (epidermal growth factor, Gibco), 10 ng/ml LIF (leukemia inhibitory factor, Invitrogen) and 20 ng/ml bFGF (basic fibroblast growth factor, Invitrogen). The formation of spheroids was observed by images in 1 hour and 24 hours using a phase difference microscope (LumaScope 620; Etaluma).

9-2: Result

In the wound healing assay, it was confirmed that cancer cells isolated from SKOV3 spheroids prepared by culturing in pV4D4 for 8 days migrated faster than 2D-cultured control cells and filled the gap (FIG. 9a), and in the transwell-based invasion assay, the cancer cells isolated from the spheroids could penetrate the gel substrate more than the control cells (˜4 times) (FIG. 9b), and through this, it can be seen that the spheroids prepared by culturing in pV4D4 have enhanced cell mobility and penetrability.

### Example 10: Confirmation of Maintenance of CSC Characteristics of Prepared Cancer Stem Cell Spheroids

By culturing cancer cells in conventional TCPs, which is isolated from SKOV3 cancer stem cell spheroids prepared by culturing in pV4D4 for 8 days to single cells, “spheroid formation ability” was evaluated. The drawing confirming formation of spheroids by the SKOV3-ssiCSCs and U87MG-ssiCSCs was shown in FIG. 10.

As can be seen in FIG. 10, it is shown that spheroids are formed simultaneously, and thus this shows that the spheroids maintain CSC-like characteristics.

### Example 11: Confirmation of Drug Resistance of ssiCSC

One of other important characteristics of CSC is having immanent or acquired drug resistance for chemotherapeutic agents due to the ability of pushing drugs out. Regarding this, the drug-release ability of each cancer cell isolated from spheroids prepared by culturing on the pV4D4 surface for 8 days was confirmed through Hoechst-dye-based side-population assay. As a result, it was confirmed that fractions of the drug release-positive cell were significantly increased in the ssiCSC prepared from 4 kinds of cancer cell lines compared to the 2D-cultured control group. Specifically, the drug release-positive fractions were increased 0% to 13.8% in SKOV3 cell, 0.59% to 9.6% in MCF-7 cell, 0.58% to 9.2% in Hep3B cell, and 0.1% to 10% in Hep3B cell (FIG. 12a).

In addition, the drug resistance of ssiCSC for doxorubicin (DOX) known as an anticancer agent was confirmed. Specifically, ssiCSC spheroids prepared by culturing on the pV4D4 surface for 8 days were isolated to single cells, and the cell was cultured on the conventional TCP surface to a 2D single layer, and then DOX at various concentrations was treated for 24 hours. As the result of measuring the cell viability using WST-1 analysis method, ssiCSC had higher resistance even to Dox of 50 μM compared to the 2D control group (FIG. 12b). Furthermore, SKOV3- and SW480-ssiCSC had complete resistance to Dox, and SW480-ssiCSC showed higher cell viability than the cancer cells of the control group in which DOX was not treated. The SW480-ssiCSC maintained drug resistance when subcultured on the TCP surface twice, and through this, it can be seen that original cancer cells were transformed into CSC-like cells (FIG. 12c).

The drug-release ability is known to be mediated by ATP-binding cassette (ABC) protein family. Accordingly, using qRT-PCR, in SKOV3-ssiCSC, the expression of multi-drug resistance (MDR) genes, the ABCB1, ABCB2, ABCB5, ABCC1 and ABCG2 panel was analyzed. It was confirmed that in the 5 all MDR-related genes, compared to the 2D-cultured control group, ssiCSC was highly upregulated. In particular, in case of ABCB1 and ABCB5 genes, the level of upregulation was remarkable (FIG. 12d). The result that MDR genes were significantly upregulated in ssiCSC showed the correlation with the side-population assay result (FIG. 12a) and DOX resistance test result (FIG. 12b).

As the result of synthesizing molecular or functional analysis of ssiCSC spheroids of the 4 kinds of type cells, it was confirmed that cancer cells were transformed into CSC-like cells which strongly expressed CSC-related genes and had intensive drug resistance, when exposed to a specific stimulus present on the pV4D4 surface.

### Example 12: Confirmation of In Vivo Cancer-Formation Ability of ssiCSC Spheroids

The cancer-formation ability of ssiCSC in vivo was confirmed. Specifically, SKOV3-derived ssiCSC spheroids were isolated to single cells, and the cells at a series of different concentrations (10² to 10⁶ cells) were mixed with Matrigel and subcutaneously injected to BALB/c nude mice (FIG. 13a). The heterologous tumor formation by the cells isolated from the spheroids were monitored for 120 days and compared with the 2D TCP-cultured SKOV3 control group (Table 3).

TABLE 3
I Tumor formation and metstasis
of SKOV3 in BALB/c nude mice.^{a}

| | |
|---|---|
| Tumor formation | Liver metastasis |

| Cell number^{b} | 2D control | ssiCSC | 2D control | ssiCSC |
|---|---|---|---|---|
| 100 | 0/5 | 0/5 | 0/5 | 4/5 |
| 1,000 | 0/5 | 1/5 | 0/5 | 4/5 |
| 10,000 | 0/5 | 4/5 | 0/5 | 4/5 |
| 100,000 | 0/5 | 3/5 | 0/5 | 5/5 |
| 1,000,000 | 2/4 | — | 0/4 | — |

^{a}Tumor formation and mestasis were monitored up to 120 days.
^{b}All cells were dissociated into single cells and counted with a hemocytometer before subcutaneous injection.

As a result, it was confirmed that the 2D control group did not form tumor at a cell dose of 10⁵ or less (0/5 mouse), and could form tumor at 50% frequency at a cell dose of 10⁶ (2/4 mice) (Table 3). In contrast thereto, ssiCSC-derived cells could form tumor at higher frequency than the control group even at a very small dose. Specifically, the tumor-forming frequency was 60% (3/5 mice) in case of 10⁵ cell dose, 80% (4/5 mice) in case of 10⁴ cell dose, and 20% (1/5 mouse) in case of 10³ cell dose (Table 3). Considering how difficult to obtain heterologous tumor of human ovarian cells (SKOV3) from athymic nude mice without using severe combined immunodeficiency (SCID) mice in general, it could be confirmed that the cancer-formation ability of SKOV3-ssiCSC in vivo was excellent through the result.

In addition, metastatic nodules which were markedly abnormal were found in the liver of ssiCSC-inoculated mice, whereas the liver of 2D SKOV3 control group-inoculated mice appeared normal (FIG. 13b). Through histological analysis, while it was confirmed that a number of metastatic lesions appeared throughout the tissue, clearly distinguishing between the normal region and tumor region, in the ssiCSC-inoculated abnormal liver, there was no evidence of metastasis in the liver of 2D control cancer cells-inoculated mice (FIG. 13c). In particular, the mice in which cells derived from SKOV3-ssiCSC were inoculated at a cell dose of 10² showed liver metastasis at a high frequency (4/5 mice) (FIG. 13d, Table 3), and based on this, it could be confirmed that SKOV3-ssiCSCs had very enhanced metastasis ability and cancer-formation ability. The immunohistochemical examination of liver metastasis for expression of tenascin-C (TNC) which was a major component of cancer-specific ECM and an essential component of metastatic environment confirmed that TNC was significantly present around the tumor boundary in which the normal tissue was contacted (FIG. 13e). Through this, it can be seen that the tumor nodules of the liver are due to metastasis of SKOV3-ssiCSCs injected subcutaneously.

Then, the cancer-formation ability of ssiCSCs derived from various cancer cell lines was confirmed. As a result, ssiCSCs derived from luciferase-introduced MCF-7 (MCF7-Luc) cell and U87MG human glioblastoma cell had significantly increased cancer-formation ability compared to the 2D-cultured control cell (Tables 4 and 5).

TABLE 4
I Tumor formation of MCF-7-Luc in BALB/c nude mice.^{a}

| Cell number | 2D control | ssiCSC |
|---|---|---|
| 100 | — | 0/5 |
| 1,000 | — | 2/5 |
| 10,000 | — | 2/5 |
| 100,000 | 0/5 | 4/5 |
| 1,000,000 | 0/5 | — |
| 10,000,000 | 1/5 | — |

^{a}Tumor formation was monitored up to 90 days.

TABLE 5
I Tumor formation of U87MG in BALB/c nude mice.^{a}

| Cell number | 2D control | ULA | ssiCSC |
|---|---|---|---|
| 100 | — | 0/5 | 1/5 |
| 1,000 | — | 0/5 | 2/5 |
| 10,000 | 1/4 | 0/5 | 3/5 |
| 100,000 | 2/4 | — | — |
| 1,000,000 | 4/4 | — | — |

^{a}Tumor formation was monitored up to 90 days.

Specifically, the 2D-cultured MCF7-Luc cell did not form tumor even if inoculated at a cell dose of 10⁶ per mouse, but the MCF7-Luc-ssiCSC formed tumor at a high frequency (4/5 mice) even if inoculated at a cell dose of 10⁵ per mouse (Table 4). Similar thereto, when U87MG-ssiCSCs were inoculated at a cell dose of 10⁴, tumor was formed at 60% frequency (3/5 mice), whereas there was no tumor formed when U87MG spheroids cultured on the ULA surface were inoculated, and this shows that the difference of the cancer-formation ability of spheroids cultured in ULA- and pV4D4- is distinct.

Taking the result together, it can be seen that the pV4D4-based PTF may be used as a platform capable of preparing cancer-forming spheroids and may be used for preparation of various human heterologous tumor models which are difficult to be prepared in athymic nude mice.

### Example 13: Confirmation of Cancer-Formation Ability and Wnt/β-Catenin Signaling of ssiCSC Spheroids

To confirm cellular and molecular mechanisms related to stem cell-like characteristics of ssiCSCs, several important signaling pathways related to the cancer-formation ability and stem cell of CSCs like Notch, Hedgehog and Wnt/β-catenin were confirmed.

At first, an experiment to confirm whether the Wnt/β-catenin signaling pathway was activated and the expression of Wnt target genes (n=46) was increased in SKOV3-ssiCSCs was performed. As a result, it was confirmed that the expression of 30 genes of 46 Wnt/β-catenin target genes was increased 1.5 times in SKOV3-ssiCSC, and the expression of the core inhibitory factor of the Wnt signaling pathway, Dickkopf-related protein 1 (DKK1) was significantly reduced (FIG. 14a). In addition, as the result of qRT-PCR analysis in SKOV3-ssiCSC spheroids cultured for 1 day, 4 days and 8 days, it was confirmed that the expression of DKK1 mRNA was dramatically reduced (FIG. 14b), and this shows that Wnt/β-catenin signaling is activated from the initial step of spheroid formation. In addition, the qRT-PCR result showed that the reduction of DKK1 expression is directly related to the increase of the expression of AXIN2 (axis inhibition protein 2) and MMP2 (matrix metalloproteinase-2) which are downstream target genes of Wnt/β-catenin signaling (FIG. 14b). Furthermore, the qRT-PCR shows that there was no result of changes in the level of 0-catenin mRNA in ssiCSC spheroids, but the western blot analysis result shows that the phosphorylated 0-catenin protein was significantly reduced (FIG. 14c). Moreover, the result of immunostaining shows that 0-catenin is hardly present in the nuclei of 2D-cultured SKOV3 cells, but 0-catenin moves to the nuclei in ssiCSCs (FIG. 14d).

Then, upstream signals causing significant reduction of DKK1 in ssiCSC spheroids was confirmed. As a result, it was confirmed that TNC related to the liver metastasis (FIG. 13e) downregulated DKK1, thereby activating Wnt/β-catenin signaling pathways in SKOV3-ssiCSC. Accordingly, to confirm the association between TNC and DKK1, SKOV3-ssiCSC spheroids cultured for 8 days were immunostained. As a result, as TNC was sufficiently present throughout the spheroids, it was confirmed that the TNC downregulated target DKK1, thereby activating Wnt/β-catenin signaling pathways (FIG. 14e).

In addition, ssiCSC obtained from MCF-7, Hep3B and SW480 spheroids showed significant expression of TNC (FIG. 15a) together with significant reduction of DKK1 gene expression (FIG. 15b), and this shows that the same Wnt/β-catenin signaling is involved in the process of preparing ssiCSC in other cancer cells.

Taking the result together, activation of Wnt/β-catenin signaling pathways mediated by TNC-DKK1 shows that cancer cells can be converted into cancer-forming CSC-like phenotypes due to the pV4D4 surface.

### Example 14: Formation of Cancer Stem Cell Spheroids in FBS Medium with Increased Albumin Concentration

Cancer cells were cultured in a medium to which BSA was added so that the albumin concentration in the FBS medium was higher than a certain level, to confirm whether cancer stem cell spheroids were formed.

Specifically, after adding BSA to the FBS medium so that the albumin concentration was 5 mg/ml, 10 mg/ml, SKOV3 cells were cultured on the pV4D4 substrate. As a control group, an FBS medium to which BSA was not added was used.

As a result, as could be seen in FIG. 16a, it was confirmed that spheroids were not formed well when the albumin concentration was not a certain level or higher as BSA was not added, but spheroids were formed when the albumin concentration was increased at a certain level or higher as BSA was added.

In addition, as the result of measuring the expression level of DKK1 of cancer cells cultured like this and showing it based on Beta-actin (FIG. 16b) and GAPDH (FIG. 16c), it was confirmed that cancer stem cell characteristics were not shown when cultured in the FBS medium to which BSA was not added, but cancer stem cells were induced only when the albumin concentration was increased at a certain level or higher by adding BSA.

1. A method for producing cancer stem cells from cancer cells, comprising culturing cancer cells using a composition comprising albumin and a medium for cell culture.

2. The method according to claim 1, wherein the cancer stem cells are in a spheroid form.

3. The method according to claim 1, wherein the albumin is comprised in the composition at a concentration of 0.1 to 500 mg/ml.

4. The method according to claim 1, wherein the albumin is provided as a serum replacement including albumin, or as a Fetal Bovine Serum (FBS) supplemented by albumin.

5. (canceled)

6. The method according to claim 1, wherein the albumin is selected from the group consisting of bovine serum albumin, human serum albumin and combinations thereof.

7. The method according to claim 1, wherein the cancer stem cells are cancer stem cells specific to an individual who the cancer cell is derived from.

8. The method according to claim 1, wherein the cancer stem cells have at least one characteristic selected from the group consisting of strengthened or enhanced cell migration, cell penetration, drug resistance and cancer-formation ability compared to the parent cancer cells.

9. The method according to claim 1, wherein the cancer stem cells express at least one marker selected from the group consisting of CD47, BMI-1, CD24, CXCR4, DLD4, GLI-1, GLI-2, PTEN, CD166, ABCG2, CD171, CD34, CD96, TIM-3, CD38, STRO-1, CD19, CD44, CD133, ALDH1A1, ALDH1A2, EpCAM, CD90, and LGR5.

10. The method according to claim 1, wherein the cancer cells are derived from ovarian cancer, breast cancer, liver cancer, brain cancer, colorectal cancer, prostate cancer, cervical cancer, lung cancer, stomach cancer, skin cancer, pancreatic cancer, oral cancer, rectal cancer, laryngeal cancer, thyroid cancer, parathyroid cancer, colon cancer, bladder cancer, peritoneal carcinoma, adrenal cancer, tongue cancer, small intestine cancer, esophageal cancer, renal pelvis cancer, renal cancer, heart cancer, duodenal cancer, ureteral cancer, urethral cancer, pharynx cancer, vaginal cancer, tonsil cancer, anal cancer, pleura cancer, thymic carcinoma or nasopharyngeal carcinoma.

11.-13. (canceled)

14. The method according to claim 1, wherein the culturing cancer cells is performed by culturing cancer cells on a cell culture substrate comprising a cyclosiloxane polymer.

15. The method according to claim 14, wherein the cell culture substrate comprising a cyclosiloxane polymer has a water contact angle of less than 90°.

16. The method according to claim 14, wherein the cyclosiloxane polymer is a homopolymer or heteropolymer comprising the monomer having the following chemical formula 1: 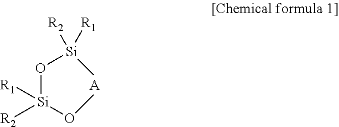
in the formula,
A is 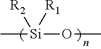
(n=an integer of 1-8); and
R1 is independently hydrogen or C2-10 alkenyl with the proviso that at least two positions of R1 are C2-10 alkenyl; and
R2 is independently of each other hydrogen, C1-10 alkyl, C2-10 alkenyl, halo group, metal element, C5-14 heterocycle, C3-10 cycloalkyl or C3-10 cycloalkenyl.

17. The method according to claim 16, wherein the compound of chemical formula 1 has n+1 or n+2 of C2-10 alkenyl at the R1 position.

18. The method according to claim 17, wherein the cyclosiloxane polymer is selected from the group consisting of 2,4,6,8-tetra(C2-10)alkenyl-2,4,6,8-tetra(C1-10)alkylcyclotetrasiloxane, 1,3,5-tri(C1-10)alkyl-1,3,5-tri(C2-10)alkenylcyclotrisiloxane, 1,3,5,7-tetra(C1-10)alkyl-1,3,5,7-tetra(C2-10)alkenylcyclotetrasiloxane, 1,3,5,7,9-penta(C1-10)alkyl-1,3,5,7,9-penta(C2-10)alkenylcyclopentasiloxane, 1,3,5-tri(C1-10)alkyl-1,3,5-tri(C2-10)alkenylcyclotrisiloxane, 1,3,5,7-tetra(C1-10)alkyl-1,3,5,7-tetra(C2-10)alkenylcyclotetrasiloxane, 1,3,5,7,9-penta(C1-10)alkyl-1,3,5,7,9-penta(C2-10)alkenylcyclopentasiloxane, 1,3,5-tri(C1-10)alkyl-1,3,5-tri(C2-10)alkenylcyclotrisiloxane, 1,3,5,7-tetra(C1-10)alkyl-1,3,5,7-tetra(C2-10)alkenylcyclotetrasiloxane, 1,3,5,7,9-penta(C1-10)alkyl-1,3,5,7,9-penta(C2-10)alkenylcyclopentasiloxane, hexa(C2-10)alkenylcyclotrisiloxane, octa(C2-10)alkenylcyclotetrasiloxane, deca(C2-10)alkenylcyclopentasiloxane, 2,4,6,8-tetravinyl-2,4,6,8,-tetramethylcyclotetrasiloxane and combinations thereof.

19. The method according to claim 16, wherein the cyclosiloxane polymer is a heteropolymer of a first monomer having chemical formula 1 and a second monomer comprising a vinyl group; and
the second monomer is at least one selected from the group consisting of siloxane having a vinyl group, methacrylate-based monomers, acrylate-based monomers, aromatic vinyl-based monomers, acrylamide-based monomers, maleic anhydride, silazane or cyclosilazane having a vinyl group, C3-10 cycloalkane having a vinyl group, vinyl pyrrolidone, 2-(methacryloyloxy)ethyl acetoacetate, 1-(3-aminopropyl)imidazole, vinyl imidazole, vinyl pyridine, and silane having a vinyl group.

20. The method according to claim 18, wherein the second monomer is at least one selected from the group consisting of 1,3,5-trivinyl-1,3,5-trimethylcyclotrisiloxane, 2,4,6,8-tetramethyl-2,4,6,8-tetravinylcyclotetrasiloxane (V4D4), 2,4,6,8,10-pentamethyl-2,4,6,8,10-pentavinylcyclopentasiloxane, 2,4,6,8,10,12-hexamethyl-2,4,6,8,10,12-hexavinyl-cyclohexasiloxane, octa(vinylsilasesquioxane), and 2,2,4,4,6,6,8,8,10,10,12,12-dodecamethylcyclohexasiloxane.

21. The method according to claim 1, wherein the method of producing cancer stem cells in a spheroid does not perform artificial gene manipulation.

22. A kit for producing cancer stem cells in a spheroid comprising,
a cell culture substrate comprising a cyclosiloxane polymer, and
a composition comprising albumin at a concentration of 0.1 to 500 mg/ml.

23. A method for screening a therapeutic drug for cancer, comprising
producing cancer stem cells by the method according to claim 1;
contacting a candidate substance to the cancer stem cell;
measuring viabilities of the cancer stem cells in the test group treated by the candidate substance and in the control group untreated by the candidate substance; and
comparing the viabilities of cancer stem cells in the group treated by the candidate substance and in the control group untreated by the candidate substance.

24. The method according to claim 23, further comprising determining the candidate substance as a therapeutic drug for cancer, when the viability of the test group is lower than that of the control group.

25. (canceled)
